(19) United States
(12) Patent Application Publication
(10) Pub. No.: US 2025/0099580 A1
(43) Pub. Date: Mar. 27, 2025

(54) TREATMENT OF GASTRIC CANCER AND/OR GASTROESOPHAGEAL JUNCTION CANCER

(71) Applicant: AstraZeneca AB, Södertälje (SE)

(72) Inventors: John KURLAND, Wilmington, DE (US); Shao-chun CHANG, Wilmington, DE (US); Gordana VLAHOVIC, Wilmington, DE (US); Osamu TAKAHASHI, Wilmington, DE (US)

(21) Appl. No.: 18/890,856

(22) Filed: Sep. 20, 2024

Related U.S. Application Data
(60) Provisional application No. 63/584,708, filed on Sep. 22, 2023.

(51) Int. Cl. A61K 39/395; A61K 31/282; A61K 31/337; A61K 31/513; A61K 31/519; A61K 39/00

(52) U.S. Cl. CPC A61K 39/3955; A61K 31/282; A61K 31/337; A61K 31/513; A61K 31/519; A61K 2039/505; A61K 2039/545

(57) ABSTRACT

The disclosure relates to methods and compositions for treating patients with resectable gastric cancer and/or gastroesophageal junction cancer using durvalumab and chemotherapy.

### FIELD OF THE DISCLOSURE

The present disclosure relates to methods and compositions for treating patients with resectable gastric and/or gastroesophageal junction cancer using durvalumab and chemotherapy.

### SEQUENCE LISTING

This application contains a sequence listing which is submitted electronically and is hereby incorporated by reference in its entirety. The sequence listing submitted herewith is contained in the XML filed created Sep. 15, 2023 entitled “23-0538-US-PRO_SequenceListing.xml” and is 8,192 bytes in size.

### BACKGROUND OF THE DISCLOSURE

Gastroesophageal junction cancer (GEJC) and gastric cancer (GC) represent the sixth most common cancer and the second most common cause of cancer death (GLOBOCAN, 2018). GC is more common in Asia, where the majority (>70%) of new cases are diagnosed, while GEJC is common in Western countries. Approximately 1 million new cases are diagnosed each year; approximately 40% of cases are diagnosed as resectable GC/GEJC.

Surgery is the primary treatment option for patients with potentially resectable tumors. However, since recurrence after surgery alone is common, a multimodality approach including systemic therapy and/or radiotherapy is recommended in combination with radical surgery and lymph node (LN) dissection to prevent recurrence. In Western countries, neoadjuvant-adjuvant chemotherapy is the preferred treatment option for patients with resectable GC/GEJC (NCCN, 2018, Smyth et al., 2016). Neoadjuvant chemotherapy (Category 2B; NCCN, 2018) or alternatively adjuvant chemotherapy (Smyth et al., 2016) alone are also options. In East Asian countries, where patients more commonly present with early stages of gastric cancer due to screening and surveillance programs, the treatment approach generally comprises surgical resection with extensive LN dissection followed by adjuvant chemotherapy (NCCN, 2018). In some Asian countries such as China and Hong Kong, high risk patients with stage III or IVA potentially resectable tumors are treated with neoadjuvant-adjuvant chemotherapy.

Neoadjuvant-adjuvant chemotherapy was first established as standard treatment in resectable GC/GEJC by the UK MRC MAGIC trial (Cunningham et al., 2006), which showed an improvement in 5-year survival from 23% to 36% for patients with resectable stage II and III GC/GEJC treated with surgery alone versus surgery plus neoadjuvant-adjuvant chemotherapy of epirubicin, cisplatin, and 5-FU [fluorouracil] [ECF]). Similar results were reported in the FNCLCC ACCORD 07 trial using neoadjuvant-adjuvant cisplatin and 5-FU (Ychou et al., 2011). Other platinum/fluoropyrimidine doublets or triplets also in use include ECX (epirubicin, cisplatin, capecitabine), EOX (epirubicin, oxaliplatin, capecitabine; Cunningham et al., 2008), and FOLFOX (5-FU+leucovorin+oxaliplatin; NCCN, 2018, Smyth et al., 2016).

A number of studies have demonstrated the clinical benefit for neoadjuvant-adjuvant treatment GC/GEJC; however, the recurrence rate within 5 years after surgery remains high and the 5-year overall survival (OS) rate remains poor, especially in Western countries. Therefore, there is a significant unmet medical need in resectable GC/GEJC and a novel therapy is globally required.

### SUMMARY OF THE DISCLOSURE

The present disclosure relates to methods for treating resectable gastric cancer (GC) and/or resectable gastroesophageal junction cancer (GEJC).

The disclosure provides a method of treating a patient identified as having resectable gastric cancer (GC) and/or resectable gastroesophageal junction cancer (GEJC), the method comprising administering to the patient a combination therapy comprising durvalumab and a chemotherapy.

The disclosure also provides a method of treating a patient identified as having resectable gastric cancer (GC) and/or gastroesophageal junction cancer (GEJC), the method comprising: (i) administering to the patient, about every 4 weeks (Q4W), about 1500 mg of durvalumab on Day 1 and a chemotherapy on Day 1 and Day 15; (ii) removing the GC and/or the GEJC by surgery; (iii) administering to the patient, about every 4 weeks (Q4W), about 1500 mg of durvalumab on Day 1 and a chemotherapy on Day 1 and Day 15; and (iv) administering to the patient about 1500 mg of durvalumab about every 4 weeks (Q4W).

The disclosure further provides a combination therapy comprising durvalumab and a chemotherapy for use in treating resectable gastric cancer (GC) and/or resectable gastroesophageal junction cancer (GEJC) in a patient in need thereof.

The disclosure further provides a combination therapy comprising durvalumab and a chemotherapy for use in treating a patient identified as having resectable gastric cancer (GC) and/or gastroesophageal junction cancer (GEJC), wherein the combination therapy comprises: (i) administering to the patient, about every 4 weeks (Q4W), about 1500 mg of durvalumab on Day 1 and a chemotherapy on Day 1 and Day 15; (ii) removing the GC and/or the GEJC by surgery; (iii) administering to the patient, about every 4 weeks (Q4W), about 1500 mg of durvalumab on Day 1 and a chemotherapy on Day 1 and Day 15; and (iv) administering to the patient about 1500 mg of durvalumab about every 4 weeks (Q4W).

The disclosure further provides use of a combination therapy comprising durvalumab and a chemotherapy for the manufacture of a medicament for treating resectable gastric cancer (GC) and/or gastroesophageal junction cancer (GEJC) in a patient in need thereof.

The disclosure further provides use of durvalumab and a chemotherapy in the manufacture of a medicament for treating resectable gastric cancer (GC) and/or gastroesophageal junction cancer (GEJC) in a combination therapy, wherein the combination therapy comprises: (i) administering to the patient, about every 4 weeks (Q4W), about 1500 mg of durvalumab on Day 1 and a chemotherapy on Day 1 and Day 15; (ii) removing the GC and/or the GEJC by surgery; (iii) administering to the patient, about every 4 weeks (Q4W), about 1500 mg of durvalumab on Day 1 and a chemotherapy on Day 1 and Day 15; and (iv) administering to the patient about 1500 mg of durvalumab about every 4 weeks (Q4W).

These and other features and advantages of the present disclosure will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS/FIGURES

The accompanying drawings are included to provide a further understanding of the methods and compositions of the disclosure. The drawings illustrate one or more embodiments of the disclosure, and together with the description serve to explain the principles and operation of the disclosure.

FIG. 1A shows the study design for the randomized, double-blind, placebo-controlled, phase III study of neoadjuvant-adjuvant durvalumab and FLOT chemotherapy followed by adjuvant durvalumab in patients with resectable gastric or gastroesophageal junction canceradenocarcinoma. BICR=blinded independent central review; GEJ=gastroesophageal junction; D1=Day 1 of the cycle; D15=Day 15 of the cycle; DFS=disease-free survival; DFS24=disease-free survival at 24 months; DFS36=disease-free survival at 36 months; DSS=disease-specific survival; Durva=durvalumab (1500 mg Q4W); ECOG=Eastern Cooperative Oncology Group; EFS=event-free survival; EFS24=event-free survival at 24 months; EFS36=event-free survival at 36 months; FLOT=fluorouracil (5-FU)+leucovorin+oxaliplatin+docetaxel (5-FU 2600 mg/m², oxaliplatin 85 mg/m², docetaxel 50 mg/m², leucovorin 200 mg/m² on Days 1 and 15 Q4W); MFS=metastasis-free-survival; OS=overall survival; OS24=overall survival at 24 months; OS36=overall survival at 36 months; pCR=pathological complete response; PD-L1=programmed cell death-ligand-1; PROs=patient-reported outcomes; PS=performance status; Q4W=every 4 weeks; R=randomization.

FIG. 1B shows a flow diagram for the randomized, double-blind, placebo-controlled, phase III study of neoadjuvant-adjuvant durvalumab and FLOT chemotherapy followed by adjuvant durvalumab in patients with resectable gastric and gastroesophageal junction cancer). Durva=durvalumab; FLOT=fluorouracil+leucovorin+oxaliplatin+docetaxel; GC=gastric cancer; GEJC=gastroesophageal junction cancer; PS=performance status; Q4W=every 4 weeks; R=randomization.

FIG. 2 shows pathological complete response (pCR-BICR) by subgroups. The data demonstrate that pCR rates are consistent across evaluable subgroups and favor durvalumab in combination with FLOT chemotherapy. Size of the circles is proportional to the number of patients. The grey band represents the 95% confidence interval for the all subjects odds ratio. Odds ratio and associated 95% confidence intervals (CIs) are calculated using unstratified Cochran-Mantel-Haenszel tests. An odds ratio>1 favours durvalumab. [a] Analysis stratified by geographic region, clinical lymph node status and PD-L1 expression status at randomization.

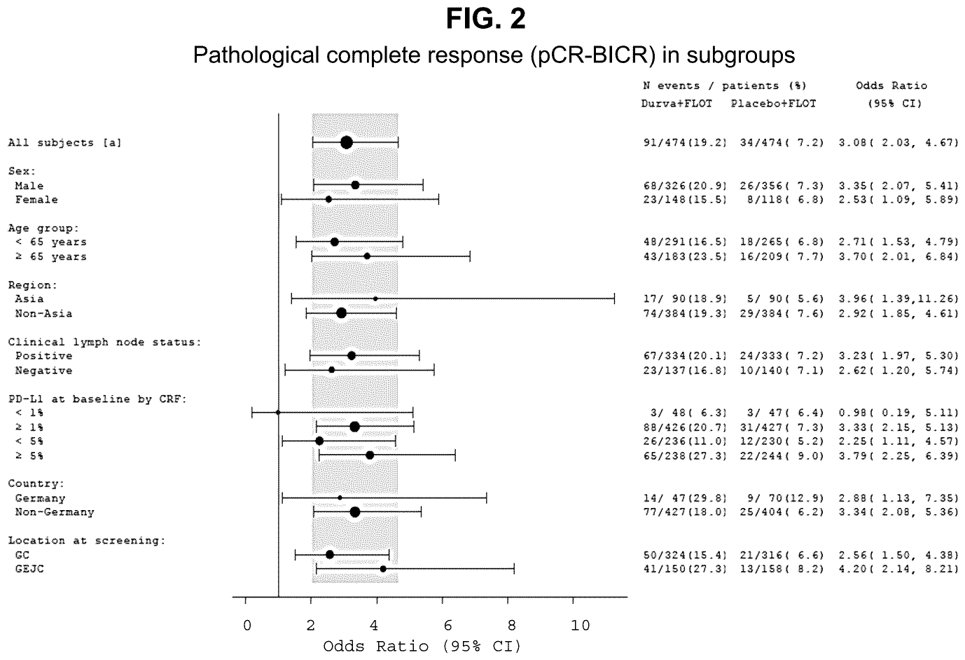

FIG. 3 shows major pathological response (complete or near-complete response BICR) by subgroups. The data demonstrate that major response rates are consistent across evaluable subgroups and favor durvalumab in combination with FLOT chemotherapy. Size of the circles is proportional to the number of patients. The grey band represents the 95% confidence interval for the all subjects odds ratio. Odds ratio and associated 95% confidence intervals (CIs) are calculated using unstratified Cochran-Mantel-Haenszel tests. An odds ratio>1 favours durvalumab. [a] Analysis stratified by geographic region, clinical lymph node status and PD-L1 expression status at randomization.

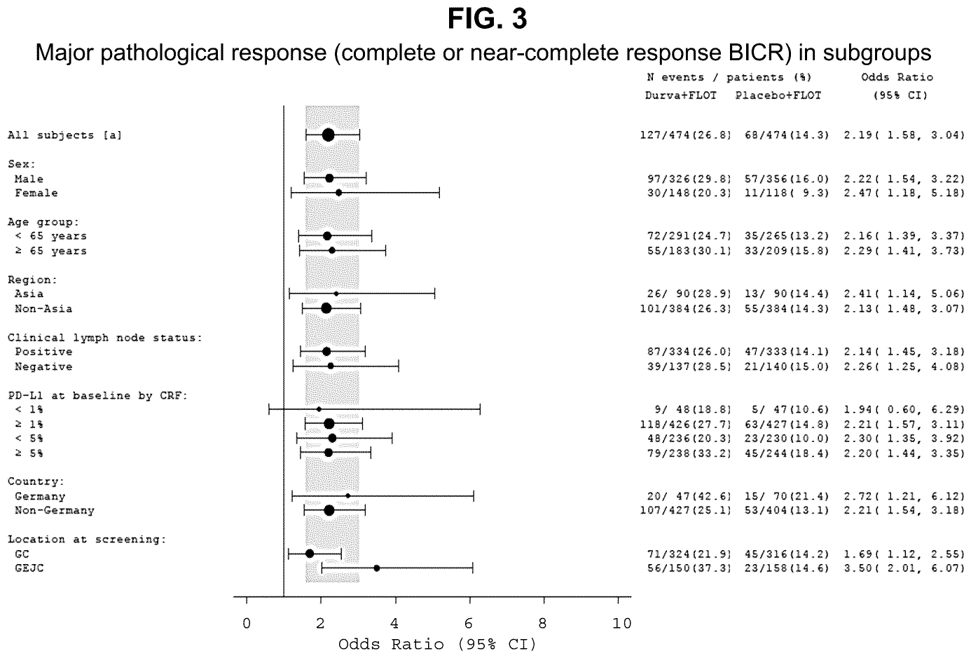

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiments of the present disclosure.

### DETAILED DESCRIPTION OF THE DISCLOSURE

The present disclosure relates to methods for treating resectable gastric cancer (GC) and/or resectable gastroesophageal junction cancer (GEJC). This disclosure demonstrates that treatment with perioperative (neoadjuvant-adjuvant) durvalumab and perioperative chemotherapy results in a significant improvement of both pathological complete response (pCR) and combined near-complete pathological complete response (pnCR) among patients with resectable GC and/or resectable GEJC, particularly after neoadjuvant treatment.

The disclosure provides a method of treating a patient identified as having resectable gastric cancer (GC) and/or resectable gastroesophageal junction cancer (GEJC), the method comprising administering to the patient a combination therapy comprising durvalumab and a chemotherapy.

The disclosure also provides a method of treating a patient identified as having resectable gastric cancer (GC) and/or gastroesophageal junction cancer (GEJC), the method comprising: (i) administering to the patient, about every 4 weeks (Q4W), about 1500 mg of durvalumab on Day 1 and a chemotherapy on Day 1 and Day 15; (ii) removing the GC and/or the GEJC by surgery; (iii) administering to the patient, about every 4 weeks (Q4W), about 1500 mg of durvalumab on Day 1 and a chemotherapy on Day 1 and Day 15; and (iv) administering to the patient about 1500 mg of durvalumab about every 4 weeks (Q4W).

The disclosure further provides a combination therapy comprising durvalumab and a chemotherapy for use in treating resectable gastric cancer (GC) and/or gastroesophageal junction cancer (GEJC) in a patient in need thereof.

The disclosure further provides a combination therapy comprising durvalumab and a chemotherapy for use in treating a patient identified as having resectable gastric cancer (GC) and/or gastroesophageal junction cancer (GEJC), wherein the combination therapy comprises: (i) administering to the patient, about every 4 weeks (Q4W), about 1500 mg of durvalumab on Day 1 and a chemotherapy on Day 1 and Day 15; (ii) removing the GC and/or the GEJC by surgery; (iii) administering to the patient, about every 4 weeks (Q4W), about 1500 mg of durvalumab on Day 1 and a chemotherapy on Day 1 and Day 15; and (iv) administering to the patient about 1500 mg of durvalumab about every 4 weeks (Q4W).

The disclosure further provides use of a combination therapy comprising durvalumab and a chemotherapy for the manufacture of a medicament for treating resectable gastric cancer (GC) and/or gastroesophageal junction cancer (GEJC) in a patient in need thereof.

The disclosure further provides use of durvalumab and a chemotherapy in the manufacture of a medicament for treating resectable gastric cancer (GC) and/or gastroesophageal junction cancer (GEJC) in a combination therapy, wherein the combination therapy comprises: (i) administering to the patient, about every 4 weeks (Q4W), about 1500 mg of durvalumab on Day 1 and a chemotherapy on Day 1 and Day 15; (ii) removing the GC and/or the GEJC by surgery; (iii) administering to the patient, about every 4 weeks (Q4W), about 1500 mg of durvalumab on Day 1 and a chemotherapy on Day 1 and Day 15; and (iv) administering to the patient about 1500 mg of durvalumab about every 4 weeks (Q4W).

In certain embodiments of the disclosure, each of steps (i) to (iv) of the methods, combination therapies for use, or uses disclosed herein is performed in succession. In certain embodiments of the disclosure, each of steps (ii) to (iv) of the methods, combination therapies for use, or uses disclosed herein is only performed after completion of the previous step. For example, step (ii) is only performed once step (i) has been completed, step (iii) is only performed once step (ii) has been completed, and step (iv) is only performed once step (iv) has been completed.

In some embodiments, step (i) is administered for about 2 cycles, step (iii) is administered for about 2 cycles, and/or step (iv) is administered for at least about 10 cycles.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps.

As used herein, the singular forms “a,” “an,” and “the” include plural referents unless the context clearly indicates otherwise. Thus, unless otherwise required by context, singular terms shall include pluralities and plural terms shall include the singular.

Percentages disclosed herein can vary in amount by +10%, 20%, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example. Additionally, “about” can mean a range of up to ±10%. When particular values or compositions are provided in the application and claims, unless otherwise stated, the meaning of “about” should be assumed to be within an acceptable error range for that particular value or composition. Unless otherwise clear from context, all numerical values provided herein are modified by the term “about.”

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the terms “treat,” “treatment,” or “treating” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on. The term “patient” may refer to a human.

In an aspect, this disclosure provides methods of treating a patient identified as having resectable gastric cancer (GC) and/or resectable gastroesophageal junction cancer (GEJC). GC, also known as stomach cancer, is a cancer that develops from the lining of the stomach. The GC referred to herein may be a gastric adenocarcinoma. GEJC is a cancer that develops at the junction of the esophagus and stomach.

The clinical stages of GC are:
Stage 0—limited to the inner lining of the stomach.
Stage I—penetration to the second or third layers of the stomach (stage 1A) or to the second layer and nearby lymph nodes (stage 1B).
Stage II—penetration to the second layer and more distant lymph nodes, or the third layer and only nearby lymph nodes, or all four layers but not the lymph nodes.
Stage III—penetration to the third layer and more distant lymph nodes, or penetration to the fourth layer and either nearby tissues or nearby or more distant lymph nodes.
Stage IV—cancer has spread to nearby tissues and more distant lymph nodes, or has metastasized to other organs.

The TNM Classification of Malignant Tumors (“TNM”), which is sometimes referred to as the AJCC/UICC staging system or the UICC/AJCC staging system, may also be used to classify the stage (i.e., anatomical extent of the spread) of GC. The outline for the TNM classification is as follows:
T—size or extent of the primary tumor
   Tx—tumor cannot be assessed
   Tis—carcinoma in situ
   T0—no evidence of tumor
   T1, T2, T3, or T4—size and/or extension of the primary tumor
N—degree of spread to regional lymph nodes
   Nx—lymph nodes cannot be assessed
   N0—no regional lymph nodes metastasis
   N1—regional lymph node metastasis present, with tumor having spread to closest or small number of regional lymph nodes at some sites
   N2—tumor has spread to an extent between N1 and N3 (N2 is not used at all sites)
   N3—tumor spread to more distant or numerous regional lymph nodes (N3 is used at all sites) not
M—presence of distant metastasis
   M0—no distant metastasis
   M1—metastasis to distant organs (beyond regional lymph nodes)

Staging of GEJC is based on the TNM staging system; a preferred classification for GEJC is the 2010 AJCC staging system for cancer. Alternatively, GEJC staging is based on the Siewert classification in which GEJC tumors are staged according to their relationship to anatomical landmarks (Siewert et al., 1996). The outline for the Siewert classification is as follows:
Type 1—adenocarcinoma of the lower esophagus with the center located within 1 cm to 5 cm above the anatomic GEJ
Type 2—true carcinoma of the cardia at the GEJ, with the tumor center within 1 cm above and 2 cm below the GEJ
Type 3—subcardial carcinoma with the tumor center between 2 and 5 cm below GEJ, which infiltrates the GEJ and lower esophagus from below.

In some embodiments, the patient having resectable GC and/or resectable GEJC is newly diagnosed. In some embodiments, the patient having resectable GC and/or resectable GEJC is previously untreated. In some embodiments, the patient having resectable GC has Stage 0, Stage I, Stage II, Stage III, or Stage IV GC. In some embodiments, the patient having resectable GC and/or resectable GEJC is documented as having radical-surgery eligible >T2 N0-3 M0 (according to the American Joint Committee on Cancer [AJCC] Cancer Staging Manual, 8th edition). In some embodiments, the patient having resectable GC and/or resectable GEJC is documented as having radical-surgery eligible >T0-4 N1-3 M0 (according to the American Joint Committee on Cancer [AJCC] Cancer Staging Manual, 8th edition). In some embodiments, the patient having resectable GC and/or resectable GEJC is newly diagnosed, previously untreated, and having resectable GC and/or resectable GEJC having radical-surgery eligible >T2 N0-3 M0 or T0-4 N1-3 M0 (according to the American Joint Committee on Cancer [AJCC] Cancer Staging Manual, 8th edition). In some embodiments, the patient having resectable GC and/or resectable GEJC is previously untreated. In some embodiments, the patient having resectable GEJC has a Siewert Type 2 and 3 tumor. In other embodiments, the patient having resectable GEJC has a Type 1 tumor that is being treated as a Siewert Type 2 and 3 tumor. In some embodiments, the patient having resectable GC and/or resectable GEJC is a candidate for gastrectomy or gastroesophagectomy (with at least D1 lymphadenectomy, preferably D2 lymphadenectomy) as planned surgery. In some embodiments, there is no evidence of metastasis in the patient having resectable GC and/or resectable GEJC. In some embodiments, the patient is lymph node positive for cancer metastasis. In some embodiments, the patient having resectable GC and/or resectable GEJC has a World Health Organization (WHO)/ECOG performance status (PS) of 0 or 1.

In some embodiments, the patient's tumor PD-L1 status of the GC and/or the GEJC is determined prior to treatment. In some embodiments, the GC tumor and/or the GEJC tumor of the patient has a PD-L1 tumor cell expression <1%. In some embodiments, the GC tumor and/or the GEJC tumor of the patient has a PD-L1 tumor cell expression ≥1%. In some embodiments, the PD-L1 expression status is either provided as a Tumor and/or Immune Cell Positivity (TIP) score or as a Tumor Area Positivity (TAP) score. In some embodiments, the PD-L1 expression status is provided as a Tumor and/or Immune Cell Positivity (TIP) score of TIP of ≥1% or a TIP of <1%. In some embodiments, the GC tumor and/or the GEJC tumor of the patient has a PD-L1 tumor cell expression 1-49%. In some embodiments, the GC tumor and/or the GEJC tumor of the patient has a PD-L1 tumor cell expression ≥50%. In certain embodiments, a patient's GC tumor and/or the GEJC tumor PD-L1 status is determined using Ventana (SP263) PD-L1 immunohistochemistry (IHC) assay applied to formalin fixed paraffin embedded tissue sample. In some embodiments, patients provide a tumor tissue sample to determine PD-L1 status using the TIP scoring method (defined as proportion of tumor area occupied by tumor cells with membrane and immune cells with cytoplasmic/membrane PD-L1 staining at any intensity) by the Ventana SP263 PD-L1 immunohistochemistry (IHC) assay for stratification in which: TIP≥1% PD-L1 is considered as high, and TIP<1% PD-L1 is considered low. In some embodiments, the PD-L1 expression status is provided as a Tumor Area Positivity (TAP) score of TAP of ≥1% or a TAP of <1%. In some embodiments, the GC tumor and/or the GEJC tumor of the patient has a PD-L1 tumor cell expression 1-49%. In some embodiments, the GC tumor and/or the GEJC tumor of the patient has a PD-L1 tumor cell expression ≥50%. The TAP score is determined on the IHC slide by visually aggregating/estimating the area covered by PD-L1 positive TC and tumor-associated IC relative to the total tumor area. The formulat for determining the TAP score is as follows: TAP=(% PD-L1 positive TC and IC)/tumour area. In certain embodiments, a patient's GC tumor and/or the GEJC tumor PD-L1 status is determined using Ventana (SP263) PD-L1 immunohistochemistry (IHC) assay applied to formalin fixed paraffin embedded tissue sample.

In some embodiments, the patient's microsatellite stability (MSI) status of the GC and/or the GEJC is determined prior to treatment. In some embodiments, the MSI status may be high or or may be low. In some embodiments, a high MSI status may be defined as having a tumour mutational burden (TMB) of ≥10 mutations per megabase. In some embodiments, the patient's MSI status is determined using a FoundationOne CDx assay (PMA P170019/S029).

As used herein, the term “durvalumab” refers to an antibody that selectively binds PD-L1 and blocks the binding of PD-L1 to the PD-1 and CD80 receptors. The durvalumab antibody is disclosed in U.S. Pat. No. 9,493,565 (referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (“ADCC”). In certain embodiments, the triple mutation refers to the IgG1 Fc region comprising a L234F/L235E/P331S triple mutation (EU numbering; see also U.S. Pat. No. 9,493,565). Durvalumab can relieve PD-L1-mediated suppression of human T-cell activation in vitro and inhibits tumor growth in a xenograft model via a T-cell dependent mechanism. Amino acid sequences of durvalumab are as follows:

TABLE 1
Durvalumab Sequences

MEDI4736 VL
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIK (SEQ ID NO: 1)
MEDI4736 VL CDR1
RASQRVSSSYLA (SEQ ID NO: 2)
MEDI4736 VL CDR2
DASSRAT (SEQ ID NO: 3)
MEDI4736 VL CDR3
QQYGSLPWT (SEQ ID NO: 4)
MEDI4736 VH
EVQLVESGGGLVQPGGSLRLSCAASGFTFSRYWMSWVRQAPGKGLEWVA
NIKQDGSEKYYVDSVKGRFTISRDNAKNSLYLQMNSLRAEDTAVYYCAR
EGGWFGELAFDYWGQGTLVTVSS (SEQ ID NO: 5)
MEDI4736 VH CDR1
GFTFSRYWMS (SEQ ID NO: 6)
MEDI4736 VH CDR2
NIKQDGSEKYYVDSVKG (SEQ ID NO: 7)
MEDI4736 VH CDR3
EGGWEGELAFDY (SEQ ID NO: 8)

In some embodiments, the combination therapy comprises administering about 1000 mg to about 2000 mg durvalumab once about every 28 days for up to about 2 cycles prior to surgery to remove the resectable GC and/or the resectable GEJC, and then about 1000 mg to about 2000 mg durvalumab once about every 28 days for up to about 12 cycles following the surgery. If a patient's weight falls to 30 kg or below [≤30 kg], the patient should receive weight-based dosing equivalent to about 20 mg/kg of durvalumab about Q4W until the weight improves to >30 kg, at which point the patient should start receiving the fixed dosing of durvalumab about 1500 mg about Q4W. In some embodiments, the combination therapy and/or the adjuvant combination therapy comprises about 1000 to 2000 mg of durvalumab. In some embodiments, the combination therapy and/or the adjuvant combination therapy comprises about 1500 mg of durvalumab.

In some embodiments, the combination therapy comprises administering about 1500 mg durvalumab once about every 28 days (Q4W) for up to about 2 cycles prior to surgery to remove the resectable GC and/or the resectable GEJC, and then about 1500 mg durvalumab once about every 28 days (Q4W) for up to about 12 cycles following the surgery.

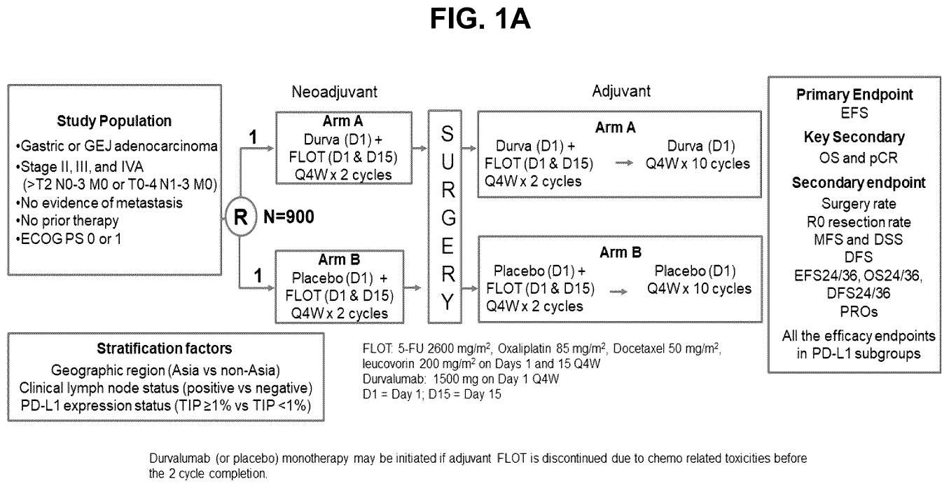

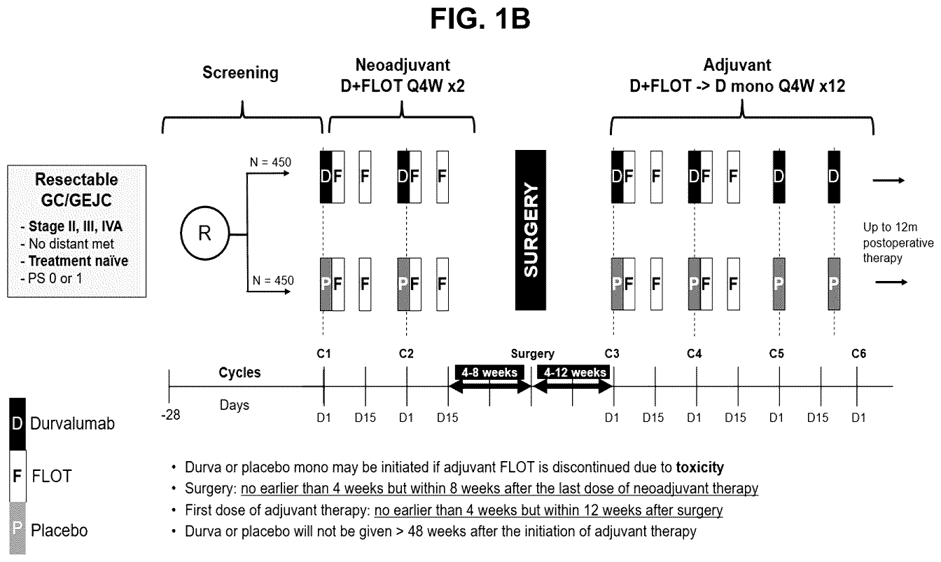

In some embodiments, the combination therapy comprises administration of 1500 mg of durvalumab on Day 1 of an about 28 day cycle, and administration of chemotherapy on Day 1 and Day 15 of the same 28 day cycle (i.e., durvalumab and the chemotherapy are both administered on Day 1, but only the chemotherapy is administered on Day 15). In some embodiments, the combination therapy is administered about every 28 days (Q4W) for a total of about 4 cycles (2 cycles neoadjuvant/before surgery and 2 cycles adjuvant/post surgery) followed by 1500 mg of durvalumab on Day 1 of an about 28 day cycle (Q4W) for about 10 additional cycles (see FIGS. 1A and 1B). In some embodiments, the combination therapy is administered for about 2 cycles. In some embodiments, on Day 1 of the combination therapy durvalumab is administered first and then the chemotherapy is administered approximately 1 to 2 hours after the end of the durvalumab infusion on Day 1 of the about 28 day cycle. In some embodiments, the combination therapy is administered about every 3 to 5 weeks. In some embodiments, the combination therapy is administered every 4 weeks.

In some embodiments, a dosage of durvalumab might range from about 0.0001 mg/kg, about 0.001 mg/kg, about 0.01 mg/kg, about 0.1 mg/kg, about 1 mg/kg, about 10 mg/kg to up to about 100 mg/kg, about 1000 mg/kg, about 10000 mg/kg or more, of the patient's body weight depending on the factors mentioned above. In some embodiments, the dosage may be between about 0.0001 mg/kg and about 20 mg/kg, about 0.0001 mg/kg and about 10 mg/kg, about 0.0001 mg/kg and about 5 mg/kg, about 0.0001 mg/kg and about 2 mg/kg, 0.0001 mg/kg and about 1 mg/kg, about 0.0001 mg/kg and about 0.75 mg/kg, about 0.0001 mg/kg and about 0.5 mg/kg, about 0.0001 mg/kg to about 0.25 mg/kg, about 0.0001 mg/kg to about 0.15 mg/kg, about 0.0001 mg/kg to about 0.10 mg/kg, about 0.001 mg/kg to about 0.5 mg/kg, about 0.01 mg/kg to about 0.25 mg/kg or about 0.01 mg/kg to about 0.10 mg/kg of the patient's body weight depending on the factors mentioned above. Typically, the clinician will administer the therapeutic antibody until a dosage is reached that achieves the desired effect.

In some embodiments, durvalumab can be provided as a 500 mg vial solution for infusion after dilution. In some embodiments, the solution contains 50 mg/mL durvalumab, 26 mM histidine/histidine-hydrochloride, 275 mM trehalose dihydrate, and 0.02% w/v polysorbate 80; it has a pH of 6.0 and density of 1.054 g/mL. In certain embodiments, a dose of about 1500 mg (for patients >30 kg in weight) can be administered using an IV bag containing 0.9% (w/v) saline or 5% (w/v) dextrose, with a final durvalumab concentration ranging from 1 to 15 mg/mL and delivered through an IV administration set with a 0.2- or 0.22-μm filter. If patient weight falls to ≤30 kg, WT-based dosing at 20 mg/kg can be administered using an IV bag selected such that the final concentration is within 1 to 15 mg/mL. In some embodiments, a standard infusion time is 1 hour (±10 minutes); however, if there are interruptions, the total allowed time must not exceed 8 hours at room temperature.

As used herein, the term “chemotherapy” refers administration of one or more anti-cancer drugs to a patient in need thereof. In some embodiments, chemotherapy refers to administration of one of one or more of fluorouracil, leucovorin, oxaliplatin, and docetaxel. In some embodiments of the methods, combinations, and uses disclosed herein, chemotherapy can refer to a combination chemotherapy, which involves treating a patient in need thereof with a number of different drugs simultaneously. For example, in some embodiments, a chemotherapy refers to chemotherapy treatment comprising at least one of one or more of fluorouracil, leucovorin, oxaliplatin, and docetaxel. In some embodiments, a combination chemotherapy can also refer to chemotherapy treatment comprising fluorouracil (5-FU), leucovorin, oxaliplatin, and docetaxel (FLOT). FLOT can be administered according to prescribing information or treatment guidance.

In certain embodiments, a chemotherapy comprises a fluorouracil dose of about 2000 to about 3000 mg/m². In certain embodiments, a chemotherapy comprises a leucovorin dose of about 175 to about 225 mg/m². In certain embodiments, a chemotherapy comprises an oxaliplatin dose of about 50 to about 100 mg/m². In certain embodiments, a chemotherapy comprises a docetaxel dose of about 25 to about 75 mg/m². In certain embodiments, a chemotherapy comprises a fluorouracil dose of about 2600 mg/m². In certain embodiments, a chemotherapy comprises a leucovorin dose of about 200 mg/m². In certain embodiments, a chemotherapy comprises an oxaliplatin dose of about 85 mg/m². In certain embodiments, a chemotherapy comprises a docetaxel dose of about 50 mg/m².

In certain embodiments, a chemotherapy comprises a fluorouracil dose of about 2600 mg/m², a leucovorin dose of about 200 mg/m², an oxaliplatin dose of about 85 mg/m², and a docetaxel dose of about 50 mg/m².

In some embodiments, the combination therapy may comprise neoadjuvant durvalumab and neoadjuvant chemotherapy. In some embodiments, the combination therapy may be perioperative durvalumab and perioperative chemotherapy. In some embodiments, the combination therapy may comprise neoadjuvant durvalumab, neoadjuvant chemotherapy, adjuvant durvalumab and adjuvant chemotherapy. In some embodiments, adjuvant durvalumab therapy may be administered to the patient after administration of adjuvant combination therapy (durvalumab and chemotherapy) has been completed. In some embodiments, adjuvant durvalumab therapy may comprise administering durvalumab to the patient after completion of treatment with adjuvant chemotherapy. As used herein, a neoadjuvant therapy refers to a treatment administered prior to a local treatment, such as surgery. As used herein, an adjuvant therapy refers to a treatment administered after a local treatment, such as radiotherapy or surgery.

In some embodiments, patients undergo resection surgery about 4 to about 8 weeks after the last dose of neoadjuvant therapy (surgery >8 weeks after the last dose of neoadjuvant therapy may be permitted). In some embodiments, adjuvant therapy begins about 4 weeks to about 12 weeks post-surgery (based on the patient's recovery period). In certain embodiments, after the surgery to remove the resectable GC and/or the resectable GEJC, patients should start the combination treatment comprising durvalumab and chemotherapy as soon as clinically feasible and within about 12 weeks from the surgery to remove the resectable GC and/or the resectable GEJC. A minimum of about 3 weeks is recommended between surgery to remove the resectable GC and/or the resectable GEJC and the start of durvalumab treatment (first post-surgical scan must be performed prior to starting adjuvant treatment and post-operative radiotherapy, if required). Complete post-operative wound healing must have occurred following any surgery.

In some embodiments, surgery to remove the resectable GC and/or the resectable GEJC should happen within about 4 weeks to about 8 weeks from the last administration of durvalumab/chemotherapy combination therapy. In some embodiments, resecting the GC and/or the GEJC occurs within about 4 weeks to about 8 weeks after the about 2 cycles of the combination therapy. In some embodiments, surgery to remove the resectable GC and/or the resectable GEJC comprises resection of the GC and/or GEJC.

In some embodiments, adjuvant combination therapy comprises administering durvalumab on Day 1 and administering chemotherapy on Day 1 and Day 15, wherein the adjuvant combination therapy is administered about every 3 to 5 weeks. In some embodiments, the adjuvant combination therapy is administered for about 2 cycles. In some embodiments, the adjuvant combination therapy further comprises administering to the patient durvalumab about every 28 days (Q4W). In some embodiments, the adjuvant combination therapy further comprises administering to the patient durvalumab about every 28 days (Q4W), for up to about 10 cycles.

In some embodiments, the success of the combination therapy is determined by pathological complete response (pCR), complete response/near-complete pathological response (pnCR), event-free survival (EFS), and/or overall survival (OS) as assessed according to RECIST 1.1 per blinded independent central review (BICR), and/or locally by pathology testing in patients with resectable GC and/or resectable GEJC.

In some embodiments, the success of the method of treatment is determined by an improvement in pathological complete response (pCR) as compared to standard of care.

In some embodiments, the success of the method of treatment is determined by an improvement in complete response/near-complete pathological response (pnCR) as compared to standard of care.

In some embodiments, the success of the method of treatment is determined by an improvement in overall survival (OS) as compared to standard of care.

In some embodiments, the success of the method of treatment is determined by an improvement event free survival (EFS) as compared to standard of care (SoC).

Thus success of treatment may be defined as an increase in pCR, pnCR, EFS, and/or OS in patients treated with the combination therapy as disclosed herein. Thus, an aspect of the disclosure may be methods, combination therapies for use, and uses disclosed herein for increasing pCR, pnCR, EFS, and/or OS, preferably compared to SoC. In some embodiments, the method, combination therapy for use, or use results in improvement in one or more of pCR, pnCR, EFS, and OS compared to a standard of care. In some embodiments, the method, combination therapy for use, or use results in improvement in one or more of pCR, pnCR, EFS, and OS compared to a neoadjuvant-adjuvant and/or adjuvant alone FLOT.

In some embodiments, the success of the method of treatment, combination therapy for use, or use is determined by an improvement in pathological complete response (pCR) by at least about 12% as compared to standard of care. In some embodiments, the success of the method of treatment, combination therapy for use, or use is determined by an improvement in combined pathological complete response/near-complete pathological response (pnCR) by at least about 13% as compared to standard of care. In some embodiments, the success of the method of treatment, combination therapy for use, or use is determined by an improvement in event-free survival (EFS). In some embodiments, the success of the method of treatment, combination therapy for use, or use is determined by an improvement in overall survival (OS) as compared to standard of care.

As used herein, the term “standard of care” (SoC) can refer to one or more of chemotherapy (preferably FLOT) or radiotherapy.

As used herein, the terms “combined pathological complete response/near-complete pathological response” or “pnCR” refer to the proportion of patients who only have single cells or rare small groups of cancer cells present in the resected specimens as determined by pathology review. In some embodiments, near-complete response indicates that single cells or rare small groups of cancer cells were present, per modified Ryan scoring system. The Ryan Scoring system is based on the ratio of residual cancer cells to the amount of fibrosis, and ranges from 1 (complete or near-complete response) to 3 (poor or not response to neoadjuvant therapy). As used herein, the terms “pathological complete response” or “pathological CR” or “pCR” refer to the proportion of patients who have no residual viable tumor in the resected specimens and as determined by pathology review. As used herein, the terms “event-free survival” or “EFS” refer the time from the date of randomization until the date of one of the following events, whichever occurs first: (1) first documented progression that precludes surgery or requires non-protocol therapy; (2) first evidence of documented recurrence of disease post-surgery in patients who had margin-negative surgery (R0); (3) first evidence of documented progression of disease post-surgery in patients with margin-positive surgery (R1/R2); or (4) death due to any cause. EFS is determined according to RECIST 1.1 per BICR assessment and/or locally by pathology testing.

As used herein, the terms “overall survival” and “OS” refer to the time from the date of randomization until date of death due to any cause, regardless of whether the patient withdraws from randomized therapy or receives another anti cancer therapy. Any patient not known to have died at the time of analysis is censored based on the last recorded date on which the patient was known to be alive. OS time equals date of OS event or censoring—date of randomization+1.

As used herein, the terms “disease-free survival” and “DFS” according to RECIST 1.1, refer to the time from the date of first post-surgery scan (i.e., Adjuvant Baseline scan) until the date of first evidence of recurrence of disease or death due to any cause, whichever occurs first, post-surgery in patients who had margin-negative surgery (R0).

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants. In certain embodiments, administration of the durvalumab and/or the chemotherapy is intravenous (IV).

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the claims in any way.

### Example 1: Neoadjuvant-Adjuvant Durvalumab and FLOT Chemotherapy Followed by Adjuvant Durvalumab in Patients with Resectable Gastric and Gastroesophageal Junction Cancer (GC/GEJC)

A randomized, double-blind, placebo-controlled, multicenter, global Phase III study is conducted to assess the efficacy and safety of neoadjuvant-adjuvant durvalumab in combination with FLOT chemotherapy followed by adjuvant durvalumab monotherapy in patients with resectable GC/GEJC (i.e., radical-surgery eligible; >T2 N0-3 M0 or T0-4 N1-3 M0, per AJCC 8th edition). Patients were randomized in a 1:1 ratio to each of the following treatment arms (see FIGS. 1A and 1B):
Treatment Arm A: durvalumab 1500 mg on Day 1+FLOT on Days 1 and 15 of Q4W, both agents being adminstered for 4 cycles (2 cycles neoadjuvant+2 cycles adjuvant) followed by durvalumab 1500 mg on Day 1 Q4W for 10 additional cycles.
Treatment Arm B: placebo on Day 1+FLOT on Days 1 and 15 of Q4W, both agents being adminstered for 4 cycles (2 cycles neoadjuvant+2 cycles adjuvant) followed by placebo on Day 1 Q4W for 10 additional cycles.

This study plans to enroll and screen approximately 1,125 patients in order to randomize approximately 900 patients in a 1:1 ratio to either Arm A or Arm B. The 5% alpha (2-sided) is split between analyses of pCR (0.1%) and EFS (4.9%). The 4.9% (2 sided) alpha allocation for the EFS analysis is controlled at interim (IA) and final analysis (FA) timepoints by using the Lan-DeMets (Lan et al., 1983) spending function that approximates the O'Brien-Fleming approach, where the significance level applied depends upon the actual proportion of information (i.e., information fraction) available. With approximately 461 EFS events at the final analysis, the study has approximately 85% power to show a statistically significant difference in EFS at the 4.20% significance level (2-sided) if the assumed true treatment effect is a HR of 0.75 for the primary treatment comparison of Arm A versus Arm B; this translates to an approximate 10-month improvement in median EFS over an assumed 30 month median EFS for Arm B, assuming EFS is exponentially distributed.

Patients were stratified according to geographic region (Asia versus non-Asia), clinical lymph node status (positive versus negative), and PD-L1 expression status (TIP/TAP≥1% versus TIP/TAP<1%). Patients provided a tumor tissue sample at screening (newly acquired or archived sample <3 months old) to determine PD-L1 status using the Tumor and/or Immune Cell Positivity (TIP) scoring method (defined as proportion of tumor area occupied by tumor cells with membrane and immune cells with cytoplasmic/membrane PD-L1 staining at any intensity) by the Ventana SP263 PD-L1 immunohistochemistry (IHC) assay for stratification in which: TIP/TAP≥1% PD-L1 is considered as high, and TIP/TAP<1% PD-L1 is considered low.

Neoadjuvant therapy begins following completion of the screening period/randomization, and patients undergo resection surgery about 4 to about 8 weeks after the last dose of neoadjuvant therapy (surgery >8 weeks after the last dose of neoadjuvant therapy may be permitted). Adjuvant therapy begins about 4 weeks to about 12 weeks post-surgery (based on the patient's recovery period). In this Phase III study, the administration of durvalumab and a FLOT chemotherapy prior to surgery, followed by further administration of durvalumab and a FLOT chemotherapy after surgery, was investigated to determine if this therapy regimen can improve activity and therefore long-term clinical outcomes for patients with resectable GC and/or resectable GEJC.

### Patients

This study enrolled and randomized approximately 900 patients at approximately 180 sites globally. Eligible patients were randomized with equal probability to 1 of 2 treatment arms, as discussed above.

Disease characteristics at screening/enrollment: histologically documented gastric or gastroesophageal junction adenocarcinoma with resectable disease (i.e., radical-surgery eligible; Stage II or higher [>T2 N0-3 M0 or T0-4 N1-3 M0] per AJCC 8th edition). GEJC includes Siewert Type 2 and 3 tumors. Siewert Type 1 tumors were eligible as long as the patient is intended to be treated in the same way as for Siewert Type 2 and 3 tumors. Per the judgment of the Investigator, the patient must be medically fit for treatment with neoadjuvant FLOT therapy prior to radical surgery. At screening, complete surgical resection of the primary GC/GEJC must be deemed achievable, as assessed by a multidisciplinary evaluation, which must include a board certified GI surgeon. No prior anti-cancer therapy (e.g., chemotherapy, radiation therapy, or chemoradiation therapy) for the current malignancy.

Patients had a World Health Organization (WHO)/ECOG performance status (PS) of 0 or 1 at enrollment.

Patients had adequate organ and marrow function as defined by (a) Hemoglobin≥9.0 g/dL; (b) Absolute neutrophil count ≥1.5×10⁹/L; (c) Platelet count ≥100×10⁹/L; (d) Serum bilirubin≤1.5×the upper limit of normal (ULN; this did not apply to patients with confirmed Gilbert's syndrome, who were allowed in consultation with their physician); (e) ALT and AST≤2.5×ULN; (f) Measured creatinine clearance (CL)>50 mL/min or Calculated creatinine clearance (CL)>50 mL/min as determined by Cockcroft-Gault (using actual body weight).

$$\begin{matrix}Males : & Creatine CL = & Weight ( kg ) \times ( 140 - Age ) \\ & ( mL / min ) & 72 \times serum creatinine ( mg / dL )\end{matrix}$$

$$\begin{matrix}Females : & Creatine CL = & Weight ( kg ) \times ( 140 - Age ) & \times 0.85 \\ & ( mL / min ) & 72 \times serum creatinine ( mg / dL ) & \end{matrix}$$

Patients must have had a life expectancy of at least 24 weeks and a body weight >30 kg at enrollment and randomization.

Tumor Sample Requirements: Tumor PD-L1 status, was confirmed by a reference laboratory using the Ventana SP263 PD L1 IHC assay, prior to randomization. As such, all patients underwent a new tumor biopsy during screening or to provide an available tumor sample taken <3 months prior to enrollment. The tumor specimen submitted to establish eligibility should be of sufficient quantity to allow for PD-L1 IHC and is preferred in formalin-fixed paraffin embedded blocks.

Patients with peritoneal dissemination (including tumor cells in peritoneal fluid) or distant metastasis were excluded. Patients with adenosquamous cell carcinoma, squamous cell carcinoma, or GI stromal tumor were excluded. Patients with a history of allogeneic organ transplantation were excluded. Patients with active or prior documented autoimmune or inflammatory disorders (including inflammatory bowel disease [e.g., colitis or Crohn's disease], diverticulitis [with the exception of diverticulosis], systemic lupus erythematosus, Sarcoidosis syndrome, or Wegener syndrome [granulomatosis with polyangiitis, Graves' disease, rheumatoid arthritis, hypophysitis, uveitis]) were excluded (with the following are exceptions patients with vitiligo or alopecia, hypothyroidism stable on hormone replacement, any chronic skin condition that does not require systemic therapy, patients without active disease in the last 5 years may be included but only after consultation with the Sponsor, and patients with celiac disease controlled by diet alone).

Additional exclusion criteria included uncontrolled intercurrent illness, including but not limited to, ongoing or active infection, symptomatic congestive heart failure, uncontrolled hypertension, unstable angina pectoris, uncontrolled cardiac arrhythmia, active interstitial lung disease, serious chronic GI conditions associated with diarrhea, or psychiatric illness/social situations that would limit compliance with study requirement, substantially increase risk of incurring AEs or compromise the ability of the patient to give written informed consent.

Patients with a history of another primary malignancy were excluded except for malignancy treated with curative intent and with no known active disease ≥5 years before the first dose of IP and of low potential risk for recurrence; adequately treated non-melanoma skin cancer or lentigo maligna without evidence of disease; and adequately treated carcinoma in situ without evidence of disease.

Patients with a history of active primary immunodeficiency or active infection including tuberculosis (clinical evaluation that includes clinical history, physical examination and radiographic findings, and tuberculosis testing in line with local practice), hepatitis B (known positive HBV surface antigen (HBsAg) result), hepatitis C, or human immunodeficiency virus (positive HIV 1/2 antibodies) were excluded. Patients with a past or resolved HBV infection (defined as the presence of hepatitis B core antibody [anti-HBc] and absence of HBsAg) were eligible. Patients positive for hepatitis C (HCV) antibody were eligible only if polymerase chain reaction is negative for HCV RNA.

Patients with any concurrent chemotherapy, IP, biologic, or hormonal therapy for cancer treatment were excluded; however, concurrent use of hormonal therapy for non-cancer-related conditions (e.g., hormone replacement therapy) is acceptable.

Patients with prior immune-mediated therapy including, but not limited to, other anti-cytotoxic T lymphocyte-associated antigen 4 (CTLA-4), anti-PD-1, anti-PD-L1, and anti programmed cell death ligand 2 (anti-PD-L2) antibodies, excluding therapeutic anticancer vaccines were excluded.

Patients must not have had current or prior use of immunosuppressive medication within 14 days before the first dose of durvalumab. The following are exceptions to this criterion: (a) Intranasal, inhaled, topical steroids, or local steroid injections (e.g., intra articular injection); (b) Systemic corticosteroids at physiologic doses not to exceed 10 mg/day of prednisone or its equivalent; (c) Steroids as premedication for hypersensitivity reactions (e.g., CT scan premedication).

### Treatments and Treatment Duration

Eligible patients received 1500 mg durvalumab or placebo on Day 1 in combination with FLOT chemotherapy on Days 1 and 15 for 2 cycles Q4W as neoadjuvant therapy. Patients completed the full 2 cycles of FLOT chemotherapy in combination with durvalumab (or placebo) before proceeding to surgery. Following surgery, patients received 1500 mg durvalumab (or placebo) in combination with FLOT chemotherapy for 2 cycles Q4W as adjuvant therapy. After 2 cycles of combination adjuvant therapy, patients received durvalumab (or placebo) monotherapy Q4W for up to a maximum of 12 cycles (including 2 cycles of durvalumab/placebo+FLOT chemotherapy and 10 additional cycles of durvalumab/placebo monotherapy).

Patients received 2 cycles of neoadjuvant therapy with 1500 mg durvalumab (or placebo) via IV infusion Q4W on Day 1 of each cycle (see FIG. 1B). Adjuvant therapy may begin 4 to 12 weeks post-surgery. Patients must have sufficiently recovered before beginning adjuvant therapy. Imaging was performed before adjuvant therapy was initiated.

Patients received adjuvant therapy with 1500 mg durvalumab (or placebo) Q4W for up to a maximum of 12 cycles (including 2 cycles of durvalumab/placebo+FLOT and 10 additional cycles of durvalumab/placebo monotherapy), with the last administration at Adjuvant Week 44, or until progression or recurrence of disease, unless there is unacceptable toxicity, withdrawal of consent, or another discontinuation criterion is met. In case of treatment delays, durvalumab/placebo treatment did exceed 12 months from the first dose of adjuvant therapy (regardless of the number of cycles of treatment received).

If a patient's weight falls to 30 kg or below (≤30 kg), the patient should receive weight-based dosing equivalent to 20 mg/kg of durvalumab Q4W until the patient's weight improves to >30 kg, at which point the patient should start receiving the fixed dosing of durvalumab 1500 mg Q4W.

### FLOT Chemotherapy

Patients received 2 cycles of neoadjuvant therapy with FLOT via IV infusion on Day 1 and 15 of each cycle. During the combination portion of treatment, durvalumab was administered first; then the FLOT chemotherapy infusion started approximately 1 hour (maximum 2 hours) after the end of the durvalumab infusion. If there were no clinically significant infusion reactions with the first cycle, then for all other cycles (including neoadjuvant and adjuvant treatment), the FLOT chemotherapy was given immediately after the durvalumab infusion had finished.

Patients completed full 2 cycles of FLOT chemotherapy in combination with durvalumab (or placebo) before proceeding to surgery. Following surgery, patients received 2 cycles of adjuvant therapy with FLOT chemotherapy on Day 1 and 15 of each cycle.

TABLE 2
Treatments

| Treatment: | Durvalumab | Placebo | FLOT Chemotherapy |
|---|---|---|---|
| Dosage | 500-mg vial | Vial | 5-FU 2600 mg/m² |
| formulation: | solution for | solution for | Leucovorin 200 mg/m² |
| | infusion after | infusion after | Oxaliplatin 85 mg/m² |
| | dilution, 50 | dilution | Docetaxel 50 mg/m² |
| | mg/mL | | vial solution for |
| | | | infusion after dilution, |
| | | | 20 mg/mL |
| Administration | IV | IV | IV |
| Route | | | |
| Dosing | 1500 mg Day 1 | Day 1 Q4 W | Days 1 and 15 of each |
| | Q4 W for 2 | for 2 cycles | 4-week cycle for 2 |
| | cycles as | Day 1 Q4 W | cycles as neoadjuvant |
| | neoadjuvant | for a | therapy Days 1 and 15 |
| | therapy | maximum of | Q4 W for 2 cycles |
| | 1500 mg Day 1 | 12 cycles | as adjuvant therapy |
| | Q4 W for a | | |
| | maximum of | | |
| | 12 cycles as | | |
| | adjuvant therapy | | |
| Packaging | Strudy treatment | Study treatment | Study treatment will |
| and labelling | will be provided | will be provided | be provided and |
| | in 500-mg vials. | in vials. Each | labeled in accordance |
| | Each 500-mg | vial will be | with regional |
| | vial will be | labelled in | requirements.^{a} |
| | labelled in | accordance with | |
| | accordance with | GMP Annex 13 | |
| | GMP Annex 13 | and per country | |
| | and per country | regulatory | |
| | regulatory | requirement. | |
| | requirement. | | |

FLOT: Fluorouracil + leucovorin + oxaliplatin + docetaxel.
GMP: Good Manufacturing Practice.
IV: Intravenous.
Q4 W: Every 4 weeks.

### Surgery

Surgical eligibility was assessed after neoadjuvant treatment and within 28 days prior to surgery. Surgery performed was gastrectomy or gastroesophagectomy with at least D1 lymphadenectomy (D2 lymphadenectomy recommended), as determined by the attending surgeon based on the intraoperative findings. Surgery was no earlier than 4 weeks but within 8 weeks after the last dose of neoadjuvant therapy. Patients must have recovered from all acute, reversible toxic effects from FLOT chemotherapy (excluding alopecia) and durvalumab (or placebo) that could potentially adversely impact the surgical procedure.

A contrast enhanced CT/MRI scan of chest, abdomen (including the entire liver and both adrenals), and pelvis were required for RECIST 1.1 assessment and for surgical planning prior to surgery. A supplemental (whole body) 18F-Fluoro-deoxyglucose positron emission tomography/computed tomography (FDG-PET)/CT scan may also be acquired prior to surgery in order to help identify distant metastasis to serve as a comparator for later PET scans to identify new lesions.

If preoperative CT/MRI and/or FDG-PET/CT were suspicious for distant metastasis or peritoneal dissemination, it was recommended that biopsy, fine needle aspiration, or staging laparoscopy was performed if those were not performed at screening and/or according to the multidisciplinary evaluation.

Complete surgical resection of the primary GC/GEJC must have been deemed achievable, as assessed by a multidisciplinary evaluation, which must include a GI surgeon who should perform gastric cancer surgery as a prominent part of his/her practice.

Radical gastrectomy or gastroesophagectomy was performed for patients who met the surgery eligibility criteria by a board certified surgeon no earlier than 28 days (4 weeks) but within 56 days (8 weeks) after the last dose of neoadjuvant therapy. (Surgery >8 weeks after the last dose of neoadjuvant therapy may be permitted in consultation with the Sponsor). The recommendations of ESMO and NCCN for the resectability of GC/GEJC was followed. The surgery was be performed by a board certified gastric surgeon, who should perform gastric surgery as a prominent part of his/her practice.

T1b-T3: adequate gastric resection to achieve negative microscopic margins (typically ≥4 cm from gross tumor).
Distal gastrectomy
Subtotal gastrectomy
Total gastrectomy
Gastroesophagectomy
T4 tumors require en bloc resection of involved structures.

Gastric resection should include the regional lymphatics—at least perigastric lymph nodes (D1), but those along the named vessels of the celiac axis (D2) was strongly recommended, with a goal of examining at least 15 or greater lymph nodes. DO was not permitted.

Routine or prophylactic splenectomy was not required. Splenectomy was acceptable when the spleen or the hilum were involved. Either open surgery or laparoscopic surgery were carried out as appropriate to the expertise of the surgeon.

### Neoadjuvant Therapy

Neoadjuvant treatment was administered beginning on Day 1 of Cycle 1 and was administered for 2 cycles that are each 4 weeks in duration (i.e., 4 administrations of FLOT chemotherapy [Q2W] and 2 administrations of durvalumab [Q4W]). During the neoadjuvant treatment period, patients who had RECIST 1.1-defined radiological progression continued to the adjuvant portion of treatment in consultation with the Sponsor if the progression event does not preclude the patient from having a radical surgery. In the event progression precluded the patient from undergoing surgery or requires non-protocol therapy as adjuvant treatment, IP was discontinued and the patient proceeded to follow-up.

### Adjuvant Therapy

Following surgery, patients received adjuvant durvalumab (or placebo) in combination with FLOT chemotherapy followed by durvalumab (or placebo) monotherapy administration eligibility must be assessed within 12 weeks from surgery, prior to starting adjuvant treatment. Patients must have received radical surgery (gastrectomy or gastroesophagectomy with at least D1 lymphadenectomy, D2 recommended). Patients must have recovered from all acute, reversible toxic effects from previous treatments that could potentially adversely impact further administration of durvalumab (or placebo)+FLOT followed by durvalumab (or placebo) monotherapy according to the Investigator's judgement. Patients should be able to start adjuvant treatment as soon as clinically feasible (no earlier than 4 weeks but within 12 weeks from surgery). Patients who recovered from treatment and/or surgery >12 weeks from surgery may be allowed in consultation with the Sponsor. The first post-surgical tumor assessment scan must be performed prior to starting adjuvant treatment. Patients who required re-resection according to Investigator's judgement were not be allowed to receive adjuvant treatment with durvalumab (or placebo)+FLOT followed by durvalumab (or placebo). No retreatment or treatment through progression was permitted in this study. During the adjuvant treatment period, patients with progression/recurrence discontinued IP. All patients who discontinued IP for any reason did proceed to follow-up.

### Endpoints and Assessments

### Efficacy Assessments

This study evaluated the primary endpoint of EFS using RECIST 1.1 per BICR assessments and/or locally by pathology testing. Biopsy histopathological assessments were used for local recurrence which was not detectable by imaging.

Efficacy assessments of pCR and the R0 resection (complete resection) rate were determined by pathology review. OS and surgery rate were also collected. EFS, MFS, and DSS were determined. DFS, DFS24, and DFS36 in patients who underwent R0 resection surgery was be determined, as was event-free survival at 24 months (EFS24) and event-free survival at 36 months (EFS36) (and/or by pathology testing for EFS24 and EFS36). OS24 and OS36 was determined.

### Tumor Assessments

### RECIST 1.1 Imaging Review-Neoadjuvant Therapy Assessments:

Radiological efficacy was assessed according to RECIST 1.1. There were 2 baseline assessments, the first for the neoadjuvant period and the second for the adjuvant period. A “Neoadjuvant Baseline” scan was collected within 28 days prior to randomization for disease staging and for use as a RECIST 1.1 baseline for the post-neoadjuvant/pre-surgery follow-up scan. Scans obtained by a patient prior to screening (i.e., prior to signing the ICF) but within 28 days prior to randomization may be used as the “Neoadjuvant Baseline” scan. The pre-surgery scan following neoadjuvant treatment was performed within 4 weeks after the last dose of neoadjuvant FLOT chemotherapy. In those patients whose progression at the pre surgery scan following neoadjuvant treatment precluded surgery, collection of scans should continue according to local practice. For patients who did not have progression at the pre-surgery scan following neoadjuvant treatment and did not have surgery, scans were conducted every 12 weeks±1 week after the date of the pre-surgery scan following neoadjuvant treatment until progression/recurrence, and these additional follow up scans used the original Neoadjuvant Baseline scan for RECIST 1.1 assessments. If an unscheduled assessment was performed and the patient had not progressed, every attempt was made to perform the subsequent assessments according to the original imaging schedule.

### RECIST 1.1 Imaging Review-Adjuvant Therapy Assessments:

An “Adjuvant Baseline” scan was collected no sooner than 4 weeks after radical surgery and preferably within 28 days prior to the first date of adjuvant treatment. Ideally, the initiation of adjuvant therapy occurred as early as possible after the date of the Adjuvant Baseline scan. In most instances, no lesions were observed on the Adjuvant Baseline scans and ‘No Evidence of Disease’ was recorded for the Adjuvant Baseline RECIST 1.1 assessment; however, if any radiologically observable tumors existed in patients who had R1/R2 surgery, a new selection of RECIST 1.1 Target and Non-Target lesions was applied.

On-study adjuvant tumor assessments occurred every 12 weeks±1 week after the date of the Adjuvant Baseline scan for 2 years and then every 24 weeks±1 week thereafter relative to the date of the Adjuvant Baseline scan until progression/recurrence, the end of study, death, discontinuation/withdrawal from study, or Sponsor decision, whichever comes first. Where possible or feasible, radiological progression/recurrence was biopsy proven.

During adjuvant treatment, the imaging schedule was followed regardless of any delays in dosing. If an unscheduled assessment was performed and the patient had not progressed, every attempt was made to perform the subsequent assessments according to the original imaging schedule. For patients who discontinued treatment due to toxicity or other reasons in the absence of objective recurrence, tumor assessments continued according to the schedules of assessments.

### Pathology Review

Pathology reviews for this study were conducted for 3 purposes: (1) pathology review for determination of staging after surgery was performed by local assessment; (2) pathology review for determination of EFS as performed by the Investigator based on local requirements; and (3) pathology review for determination of pCR was performed by central and local assessment.

Formalin-fixed surgical specimen slides were submitted according to the pathology manual for central pathology review.

Images, including unscheduled visit scans, were collected on an ongoing basis and sent for storage and BICR. Guidelines for image acquisition, de-identification, storage of digital copies at the investigative site (as source documents), and transfer to the imaging CRO were provided in a separate document. Electronic image transfer from the sites to the imaging CRO was strongly encouraged.

### Clinical Outcome Assessments

A clinical outcome assessment is an assessment of a clinical outcome made through report by a clinician, a patient, or a non-clinician observer or through a performance-based assessment (FDA-NIH BEST Resource). A clinical outcome assessment may be used in clinical studies to provide either direct or indirect evidence of treatment benefit. Patient-reported outcomes (PRO) are a type of clinical outcome assessment. PRO is an umbrella term referring to all outcomes and symptoms that are directly reported by the patient. PROs have become important in evaluating effectiveness of study treatments in clinical studies and will aid in understanding of the benefit/risk evaluation (Kluetz et al., 2018a). The following PROs were administered in this study: EORTC QLQ-C30, EORTC QLQ-STO22, EORTC IL38, PRO CTCAE, patient global assessments, and EQ-5D-5L.

EORTC QLQ-C30 and QLQ-STO22+IL38: The EORTC QLQ-C30 and the QLQ-STO22+IL38 instruments were selected to evaluate effect of treatment and disease state on symptoms, impacts, and HRQOL. The EORTC instruments included commonly reported symptoms and impacts of GEJ/GC, such as abdominal pain, early satiety, reflux symptoms, difficulty swallowing/dysphagia, appetite loss, weight loss, fatigue and physical and role function.

The EORTC QLQ-C30 includes 30 items and assesses HRQOL/health status through the following 9 multi item scales: 5 functional scales (i.e., physical, role, cognitive, emotional, and social), 3 symptom scales (i.e., fatigue, pain, and nausea and vomiting), and 1 global health status/QoL scale. It also includes 6 single-item symptom/impact measures: dyspnea, insomnia, appetite loss, constipation, diarrhea, and financial difficulties (Aaronson et al., 1993).

The EORTC QLQ-STO22 includes 22 items and assesses HRQOL/health status through 5 multi-item scales: 5 symptom scales (dysphagia, pain, reflux symptoms, eating restrictions, and anxiety), 2 single-item symptom measures: dry mouth and taste, a 1-2 item measure for hair loss, and 1 single-item functional measure: body image (Blazeby et al., 2004). The EORTC QLQ-STO22 has evidence for content validity and psychometric properties (Blazeby et al., 2004, Vickery et al., 2001).

The EORTC IL38 is a 2-item questionnaire developed to assess two additional salient GEJC/GC symptoms to complement EORTC QLQ-STO22. The 2 single item questions evaluate the following symptoms: difficulty swallowing and weight loss.

PRO-CTCAE: PRO-CTCAE was developed by the NCI in recognition that collecting treatment-related symptom data directly from patients can improve accuracy and efficiency. This was based on findings from multiple studies demonstrating that physicians and nurses underestimate symptom onset, frequency, and severity in comparison with patient ratings (Basch et al., 2009, Litwin et al., 1998, Sprangers and Aaronson, 1992). To date, 78 symptoms of the CTCAE (version 4) have been identified to be amenable to patient reporting, but not all items are administered in any clinical study. Response options vary in frequency, severity, and interference with usual activities. The PRO-CTCAE will only be administered in the languages where a linguistically validated version exists.

Patient Global Assessments (PGIS, PGIC, PGI-TT): Patient global assessments were implemented to understand patient's global perception of HRQOL and aid in further exploratory analysis.

The Patient Global Impression of Symptoms (PGIS) single question included to assess how a patient perceives his/her overall severity of cancer symptoms over the past 7 days. Patients choose from response options ranging from “no symptoms” to “very severe.” The Patient Global Impression of Change (PGIC) is a single question assessing a patient's perception of global change in health status. Patients choose from response options ranging from “Much Better” to “Much Worse.”

Overall patient perspective on treatment tolerability will be assessed using the Patient Global Assessment of Treatment Tolerability (PGI-TT). This single question provides a simple overall assessment of how patients view the burden of the symptomatic adverse reactions that may be associated with their assigned treatment (Kluetz et al., 2018b).

EQ-5D-5L: The EQ-5D-5L, developed by the EuroQoL Group, is a generic questionnaire that provides a simple descriptive profile of health and a single index value for health status for economic appraisal (EuroQol, 2015). The EQ-5D-5L questionnaire comprises 5 questions that cover 5 dimensions of health (i.e., mobility, self-care, usual activities, pain/discomfort, and anxiety/depression). Respondents also assess their health today using the EQ-visual analog scale (VAS), which ranges from 0 (worst imaginable health) to 100 (best imaginable health).

Administration of electronic patient-reported outcome questionnaires: Patients perform the PRO assessments using an electronic tablet (ePRO) during clinic study visits. If there is a technical issue with the device, or the patient is unable to visit the site, the site will have access to a web-based backup. Approximately 30 minutes is required for patients to complete the questionnaires. Each site must allocate the responsibility for the administration of the ePRO instruments to a specific individual (e.g., a research nurse or study coordinator) and, if possible, assign a backup person to cover if that individual is absent.

TABLE 3
Endpoints and Assessments

| Primary objective: | Endpoint/variable: |
|---|---|
| To compare Arm A | EFS is defined as the time from |
| relative to Arm B on | randomization to the following, |
| event-free survival | according to RECIST 1.1 per |
| (EFS) | BICR assessment and/or locally |
| | by pathology testing: |
| | 1) Progression that precludes |
| | surgery or requires non-protocol |
| | therapy, 2) Local or distant |
| | recurrence or progression of |
| | disease, or 3) death due to any |
| | cause. |
| Key secondary | |
| objectives: | Endpoints/variables: |
| To compare Arm A | Overall survival is length of |
| relative to Arm B | time from randomization until |
| on overall survival | the date of death due to any |
| (OS) | cause. |
| To compare Arm A | pCR rate is defined as the |
| relative to Arm B | proportion of patients who have |
| on pathological | no residual viable tumor in the |
| complete response | resected specimens and as |
| (pCR) rate | determined by pathology review. |
| Secondary objectives | Endpoint/variable: |
| To compare Arm A | Surgery rate is the proportion |
| relative to Arm B on | of patients who undergo |
| the proportion of | gastrectomy or |
| patients who undergo | gastroesophagectomy. |
| gastrectomy or | |
| gastroesophagectomy | |
| To compare Arm A | R0 resection (complete resection) |
| realtive to Arm B | rate is the proportion of patients |
| on the rate of | who have a confirmed complete |
| complete | resection as assessed by |
| resection (R0) | pathology review. |
| To compare Arm A | MFS is the time from |
| relative to Arm B on | randomization until the |
| metastasis-free | development of metastasis using |
| survival (MFS) and | RECIST 1.1 or death due to any |
| disease-specific | cause. |
| survival (DSS) | DSS is the time from |
| | randomization until death due |
| | to GC/GEJC. |
| To compare Arm A | DFS is defined as the time from |
| relative to Arm B on | post-surgery scan until disease |
| disease-free survival | recurrence, using RECIST 1.1, |
| (DFS) in patients | or death due to any cause. |
| who undergo R0 | |
| resection surgery | |
| To compare Arm A | EFS24 and EFS36 are the |
| relative to Arm B on | proportions of patients alive and |
| EFS24, ESF36, | event-free from randomization |
| OS24, OS36, DFS24, | until 24 and 36 months, |
| and DFS36 | respectively, according to |
| | RECIST 1.1 per BICR |
| | assessment and/or locally by |
| | pathology testing. |
| | OS24 and OS36 are the |
| | proportions of patients alive at |
| | 24 and 36 months, respectively. |
| | DFS24 and DFS36 are the |
| | proportions of patients alive and |
| | recurrence-free from first post- |
| | surgery scan until 24 and 36 |
| | months, respectively, using |
| | RECIST 1.1. |
| To compare Arm A | OS, OS24, and OS36 as |
| relative to Arm B | described above. |
| on efficacy | EFS, EFS24, EFS36, MFS, and |
| endpoints by PD-L1 | DSS as described above |
| expression | DFS, DFS24, and DFS36 as |
| | described above |
| | pCR as described above |
| To compare Arm A | Time to deterioration and change |
| relative to Arm B | from baseline: |
| on disease-related | EORTC QLQ-C30: Global health |
| symptoms, impacts, | status/QoL, impacts (e.g., physical |
| and HRQoL | and role functioning), multi-term |
| | symptoms (e.g., fatigue), and |
| | single item symptoms |
| | (e.g., appetite loss) |
| | EORTC QLQ-STO22 + IL38: |
| | Multi-term symptoms |
| | (e.g., dysphagia, reflux), single- |
| | item sign/symptoms |
| | (e.g., abdominal pain, early satiety, |
| | difficulty swallowing, weight loss) |
| To evaluate the PK | PK will be assessed through the |
| of Arm A and Arm | determination of durvalumab |
| B | concentrations in serum and |
| | docetaxel concentrations in |
| | plasma. |
| To assess the | Presence of anti-drug antibody |
| immunogenicity | (ADA) to durvalumab in patients |
| of Arm A and Arm | of Arm A and Arm B |
| B | |
| Exploratory | |
| objective: | Endpoint/variable: |
| To assess patient- | PRO-CTCAE (pre-selected items |
| reported treatment | based on treatment arms) |
| tolerability using | |
| PRO-CTCAE | |
| To assess the | Patient global assessments |
| patients’ global | |
| impression of change, | |
| symptoms severity, | |
| and global treatment | |
| tolerability | |
| To explore the impact | The EQ-5D-5L health rate state |
| of treatment and | utility instrument will be used to |
| disease state on | derive health state utility based on |
| health state utility | patient-reported data. |
| using the EQ-5D-5L | |
| To investigate the | Association of TMB, MSI, and |
| relationship between | EBV with efficacy and clinical |
| tumor mutational | parameters. |
| burden (TMB), | |
| microsatellite | |
| instability (MSI), and | |
| Epstein-Barr virus | |
| (EBV) status with | |
| efficacy parameters | |
| Safety objective: | Endpoints/variables: |
| To assess the safety | Safety and tolerability will be |
| and tolerability of | evaluated in terms of AEs, vital |
| Arm A as compared | signs, clinical laboratory, and |
| to Arm B in patients | ECG |
| with GC/GEJC | Assessments related to AEs cover: |
| | Occurrence/Frequency |
| | Relationship to IP, background |
| | chemotherapy, and surgical |
| | procedure as assessed by |
| | Investigator |
| | CTC grade |
| | Seriousness |
| | Death |
| | AEs leading to discontinuation of IP |
| | Other action taken related to IP |
| | (e.g., dose interruptions, delays, or |
| | adjustments) |
| | AEs of special interest |
| | Other significant AEs |
| | Vital signs parameters include |
| | systolic and diastolic blood |
| | pressure, pulse, respiration rate, |
| | body temperature, and body weight. |
| | Assessments cover |
| | Observed value |
| | Absolute and percent change from |
| | baseline values over time |
| | Clinically significant abnormalities |
| | over time |
| | Laboratory parameters include |
| | clinical chemistry and hematology |
| | as well as urinalysis. Assessments |
| | cover |
| | Observed value |
| | Absolute and percent change from |
| | baseline valuse over time |
| | Clinically significant abnormalities |
| | in laboratory parameters over time |
| | Laboratory status, including change |
| | in abnormality |
| | (e.g., low, normal, high) from |
| | baseline to maximum on-treatment |
| | value. |
| | Laboratory status, including change |
| | in abnormality (e.g., low, normal, |
| | high) from baseline to minimum |
| | on-treatment value. |
| | Treatment-emergent changes in |
| | laboratory parameters |

### Adverse Events and Serious Adverse Events

An adverse event is the development of any untoward medical occurrence (other than progression of the malignancy under evaluation) in a patient or clinical study patient administered a medicinal product and which does not necessarily have a causal relationship with this treatment. An adverse event (AE) can therefore be any unfavorable and unintended sign (e.g., an abnormal laboratory finding), symptom (for example nausea, chest pain), or disease temporally associated with the use of a medicinal product, whether or not considered related to the medicinal product. The term AE is used to include both serious and non-serious AEs and can include a deterioration of a pre-existing medical occurrence. An AE may occur at any time, including run-in or washout periods, even if no study intervention has been administered.

Definitions of serious adverse event: A serious adverse event is an AE occurring during any study phase (i.e., run-in, treatment, washout, follow-up), that fulfils one or more of the following criteria: results in death, is immediately life-threatening, requires in-patient hospitalization or prolongation of existing hospitalization, results in persistent or significant disability or incapacity, is a congenital abnormality or birth defect, or is an important medical event that may jeopardize the patient or may require medical treatment to prevent one of the outcomes listed above.

Adverse Events (AEs) for malignant tumors reported during a study should generally be assessed as Serious AEs. If no other seriousness criteria apply, the ‘Important Medical Event’ criterion should be used. In certain situations, however, medical judgement on an individual event basis should be applied to clarify that the malignant tumor event should be assessed and reported as a Non-Serious AE. For example, if the tumor is included as medical history and progression occurs during the study, but the progression does not change treatment and/or prognosis of the malignant tumor, the AE may not fulfill the attributes for being assessed as Serious, although reporting of the progression of the malignant tumor as an AE is valid and should occur. Also, some types of malignant tumors, which do not spread remotely after a routine treatment that does not require hospitalization, may be assessed as Non-Serious; examples in adults include Stage 1 basal cell carcinoma and Stage 1A1 cervical cancer removed via cone biopsy.

### Statistical Analysis

Descriptive statistics are used for all variables, as appropriate, and are presented by treatment arm. Continuous variables are summarized by the number of observations, mean, standard deviation, median, minimum, and maximum. Categorical variables are summarized by frequency counts and percentages for each category. Unless otherwise stated, percentages are calculated out of the population total for the corresponding treatment arm.

Baseline is the last assessment of the variable under consideration prior to the intake of the first dose of IP, except for efficacy variables. For efficacy variables except DFS, baseline is defined as the last visit prior to randomization. For DFS, the baseline scan is the first scan performed after R0 resection and prior to the start of adjuvant therapy (i.e., the Adjuvant Baseline scan).

Unless otherwise specified, efficacy and PRO data are summarized and analyzed based on the FAS. DFS endpoints are summarized and analyzed based on the R0 resection analysis set. PK data is summarized and analyzed based on the PK analysis sets. ADA data is summarized and analyzed based on the ADA analysis set. Safety data is summarized on the Safety analysis set. Results of all statistical analysis are presented using a 95% CI and 2-sided p-value, unless otherwise stated.

Table 4 details which endpoints are to be subjected to formal statistical analysis, together with pre-planned sensitivity analyses, making it clear which analysis is regarded as primary for that endpoint. Note: all endpoints compare Arm A versus Arm B in all randomized patients (FAS), unless otherwise indicated.

TABLE 4
Pre-planned Statistical and Sensitivity Analyses to be Conducted

| Endpoints analyzed | Notes |
|---|---|
| EFS | Primary analysis |
| | Stratified log-rank test on data according |
| | to RECIST 1.1 per BICR assessment |
| | and/or locally by pathology testing |
| | Sensitivity analysis |
| | Stratified log-rank test on data according |
| | to RECIST 1.1 per Investigator assessment |
| | and/or locally by pathology testing |
| OS | Key secondary analysis |
| | Straitied log-rank test |
| pCR rate | Key secondary analysis |
| | Stratified Cochran-Mantel-Haenszel test |
| Surgery rate | Stratified Cochran-Mantel-Haenszel test |
| R0 resection rate | |
| MFS, DSS, DFS | Stratified log-rank test |
| EFS24, EFS36, | Hazard ratio using Kaplan-Meier estimates |
| OS24, OS36, | (95% CI) |
| DFS24, DFS36 | |
| Each scale/item | Summary and descriptive statistics |
| of EORTC, | Unadjusted change from baseline |
| QLQ-C30, and | |
| QLQ-STO22 + | |
| IL38 | |
| Key symptoms, | MMRM analysis (overall and by each |
| functions, global | visit) |
| health status/QoL | |
| of EORTC | |
| QLQ-C30 and | |
| QLQ-STO22 + | |
| IL38 | |
| Time of symptom, | Stratified log-rank test, Kaplan-Meirer |
| function, or | estimate |
| HRQoL deterioration | |
| of key PRO endpoints | |
| using EORTC | |
| QLQ-C30 and | |
| QLQ-STO22 + | |
| IL38 | |
| Improvement rates for | Logistic regression |
| key PRO endpoints | |
| (EORTC QLQ-C30 | |
| and QLQ-STO22 + | |
| IL38) | |

BICR Blinded Independent Central Review; CI confidence interval; DFS disease-free survival; DFS24 disease-free survival at 24 months; DFS36 disease-free survival at 36 months; DSS disease-specific survival; EFS event-free survival; EFS24 event-free survival at 24 months; EFS36 event-free survival at 36 months; EORTC European Organization for Research and Treatment of Cancer; HR hazard ratio; HRQoL health-related quality of life; IL38 Item Library 38, 2-item disease specific measure to complement QLQ-STO22; MFS metastasis-free survival; MMRM mixed model repeat measures; OS overall survival; OS24 overall survival at 24 months; OS36 overall survival at 36 months; pCR pathological complete response; PD L1 programmed cell death-ligand-1; PRO patient-reported outcome; QLQ-C30 30 item core quality of life questionnaire; QLQ-STO22 22-Item Gastric Cancer-Specific Quality-of-Life Questionnaire; QoL quality of life

### Event-Free Survival (EFS)

The primary endpoint, EFS according to whichever occurs first by RECIST 1.1 per BICR assessment and/or locally by pathology testing if clinically required (e.g., local recurrence detected by gastroendoscopy, peritoneal dissemination, or distant metastasis discovered during surgery) or death, is analyzed using a stratified log-rank test adjusting for the stratification variables of geographic region (Asia versus non-Asia), clinical lymph node status (positive versus negative), and PD-L1 expression status (TIP≥1% versus TIP<1%) for the generation of the p value.

The hazard ratio (HR) and its CI are estimated from a stratified Cox proportional hazards model (with ties=Efron and stratification variables), and the CI calculated using a profile likelihood approach.

Kaplan-Meier plots are presented by treatment arm. Summaries of the numbers and percentages of patients who had an EFS event, those still in survival follow-up, those lost to follow-up, and those who have withdrawn consent will be provided along with the median EFS for each treatment arm.

The assumption of proportionality of hazards in the Cox model is assessed firstly by examining plots of complementary log-log (event times) versus log (time) and, if these raise concerns, by fitting a time-dependent covariate to assess the extent to which this represents random variation. If a lack of proportionality is evident, the variation in treatment effect is described by presenting piecewise HR calculated over distinct time periods. In such circumstances, the HR can still be meaningfully interpreted as an average HR over time unless there is extensive crossing of the survival curves. If lack of proportionality is found, this may be a result of treatment-by-covariate interactions, which will be investigated.

A sensitivity analysis of EFS is performed using Investigator assessments according to RECIST 1.1 in place of BICR assessments. Additional sensitivity analyses of the primary endpoint is performed to assess possible evaluation-time bias, attrition bias, and ascertainment bias.

Subgroup analyses is conducted comparing EFS (using RECIST 1.1 per BICR assessments and/or locally by pathology testing) between Arm A and Arm B in the following subgroups of the FAS (but not limited to): sex (male versus female), age at randomization (<65 versus ≥65 years of age), geographic region (Asia versus non-Asia), clinical lymph node status (positive versus negative), or PD-L1 expression status (TIP≥1% versus TIP<1%) (for all prespecified cutoff thresholds which are defined prospectively or during the course of exploratory analysis).

The purpose of the subgroup analyses is to assess the consistency of treatment effect across expected prognostic and/or predictive factors. Forest plots are produced. Other baseline variables may be assessed if there is clinical justification or an imbalance is observed between treatment arms. No adjustment to the significance level for testing of the subgroup and sensitivity analyses are made since all these analyses are considered supportive of the analysis of EFS.

Stratified Cox proportional hazards modeling are employed to assess the effect of stratification variables on the HR estimate. A model is constructed, containing treatment and the stratification variables, to ensure that any output from the Cox modeling is likely to be consistent with the results of the stratified log-rank test. Interactions between treatment and stratification factors are tested to rule out any qualitative interaction using the approach of Gail and Simon 1985. Additionally, for each subgroup, the HR (Arm A: Arm B) and 95% CI is calculated from a Cox proportional hazards model with treatment as the only covariate. If there are too few events available for a meaningful analysis of a particular subgroup (it is not considered appropriate to present analyses where there are less than 20 events across both treatment arms in a subgroup), the relationship between that subgroup and EFS is not be formally analyzed. In this case, only descriptive summaries is provided.

### Overall Survival

OS is analyzed using the same methodology as described for the primary EFS endpoint including a stratified log-rank test, stratified Cox proportional hazards model, and Kaplan Meier plots.

Pathological Complete Response Rate The pCR rate based on central assessment is compared between Arm A and Arm B using a stratified Cochran-Mantel-Haenszel test. The stratification variables include geographic region, clinical lymph node status, and PD-L1 expression status. The effect of treatment is estimated by the odds ratio together with its corresponding 95% CI and p-value. A sensitivity analysis of pCR rate based on local assessment is also performed.

### Surgery Rate

Surgery rate is analyzed using the same methodology as described for the pCR rate.

### R0 Resection Rate

R0 resection (margin-negative resection) rate is analyzed using the same methodology as described for the pCR rate.

### Metastasis-Free Survival (MFS)

MFS is analyzed using the same methodology as described for OS.

### Disease-Specific Survival (DSS)

DSS is analyzed using the same methodology as described for OS.

### Disease-Free Survival (DFS)

DFS in the R0 resected analysis set is analyzed using the same methodology as described for OS.

EFS24, EFS36, OS24, OS36, DFS24, and DFS36 is summarized (using the Kaplan-Meier curve) and presented by treatment arm.

Each is compared between treatments by using the Kaplan-Meier estimator of the endpoint for each treatment to obtain the HR. The HR and CI is presented using the following approach (Klein et al., 2007):

The HR (Arm 1: Arm 2) is estimated as:

$$\frac{ln ( t )}{ln ( t )}$$

The variance for ln (HR) is estimated as:

$$\frac{{( t )}^{2}}{{ln}^{2} {S}_{1} ( t )} + \frac{{( t )}^{2}}{{ln}^{2} {S}_{2} ( t )} ,$$

is the variance for InS(t) derived from Greenwood's formula for the variance of survival function S(t) and can be estimated from standard software packages, where dᵢ and nᵢ refer to the number of events and patients at risk at time ti.

### Patient-Reported Outcomes (PRO)

### EORTC QLQ-C30 and QLQ-STO22+IL38

Time to deterioration (TTD) and change from baseline is evaluated.

TTD is analyzed using a stratified log-rank test as described for the primary EFS endpoint. The effect of treatment arm (Arm A) versus control arm (Arm B) is estimated by the HR together with its corresponding 95% CI and p value. Kaplan-Meier plots are presented by treatment arm. Summaries of the number and percentage of patients who have an event, as well as who were censored, are provided along with the medians for each treatment. The population for the analysis of time to global health status/QoL or function deterioration includes a subset of the FAS who have baseline scores of ≥10. The population for the analysis of time to symptom deterioration includes a subset of the FAS who have baseline scores≤90.

Additional analyses of global health status/QoL, functioning, and symptoms focuses on comparing mean change from baseline between treatment arms. The analysis population for change from baseline is the FAS and includes all randomized patients with an evaluable baseline assessment and at least 1 evaluable post baseline assessment.

Summaries of the original and the change from baseline values of each domain/item are reported by visit for each treatment arm. Graphical presentations for relevant analyses described above may also be produced as appropriate. Additional analyses may be conducted. Further details are provided in the SAP.

### PRO-CTCAE

PRO-CTCAE data is presented using summaries and descriptive statistics based on the FAS.

### Patient Global Assessments (PGIS, PGIC, PGI-TT)

Responses on the patient global assessments are summarized descriptively as number of patients and corresponding percentages for each category in the questionnaire at each visit by treatment arm.

### EQ-5D-5L

Descriptive statistics are calculated for each scheduled visit/timepoint in the study, for each study treatment, and as a total. This reports the number of patients, the number of EQ-5D questionnaires completed at each visit, and the number and proportion of patients responding to each dimension of the EQ-5D-5L. Additionally, summary statistics (e.g., n, mean, median, SD, min, and max) are reported for the EQ-5D index score and the EQ VAS score, and the change from baseline is reported for the EQ-5D index score and the EQ-VAS score. Graphical plots of the mean EQ-5D index score and EQ-VAS score, including change from baseline, and associated 95% CI by scheduled visits/timepoints in the study may be produced.

### Adverse Events

Adverse events are coded using the most recent version of the Medical Dictionary for Regulatory Activities (MedDRA). AEs are presented for each treatment group by SOC and/or PT, covering number and percentage of subjects reporting at least one event and number of events where appropriate. AEs occurring prior to start of IP, during neoadjuvant treatment, during the surgical period, during adjuvant treatment, and post-treatment are presented separately.

Full analysis set: The Full analysis set (FAS) includes all randomized patients with treatment arms assigned in accordance with the randomization, regardless of the treatment actually received. Patients who were randomized but did not subsequently receive treatment are included in the FAS. Unless otherwise specified, the FAS is used for demography summaries and all efficacy analyses, except those involving DFS endpoints.

R0 resected analysis set: The R0 resected analysis set includes all patients in the FAS who had margin-negative or R0 surgical resection following neoadjuvant treatment. Unless otherwise specified, the R0 resected analysis set is used for DFS endpoint analyses.

Safety analysis set: The safety analysis set consists of all subjects who have received at least one dose of investigational product. Erroneously treated subjects (e.g., those randomized to treatment A but actually given treatment B) are accounted for in the treatment group of the treatment they actually received. A subject who has on one or several occasions received active IP is classified as active and is accounted for the active IP treatment group.

Durvalumab PK analysis set: All patients who receive at least 1 dose of durvalumab/placebo per the protocol, for whom there is at least 1 reportable PK concentration, are included in the durvalumab PK analysis set.

Docetaxel PK analysis set: Plasma samples are collected from 10 patients from selected sites for the determination of concentration of docetaxel. The docetaxel PK analysis set includes all of those 10 patients for whom there is at least 1 reportable PK concentration.

Anti-drug antibody analysis set: The Anti-drug Antibody analysis set consists of all patients who have a non-missing baseline ADA result and at least 1 non-missing post-baseline ADA result.

Blinded Independent Central Review (BICR): All images will be collected centrally. The imaging scans will be reviewed by 2 independent radiologists using RECIST 1.1 and will be adjudicated, if required. For each patient, the BICR will define the overall visit response data (CR, PR, SD, PD, or NE) and the relevant scan dates for each timepoint (i.e., for visits where response or progression is/is not identified). If a patient has had a tumor assessment that cannot be evaluated, then the patient will be assigned a visit response of NE (unless there is evidence of progression, in which case the response will be assigned as PD). Endpoints (of EFS) will be derived from the overall visit response date and the scan dates.

### Event-Free Survival

EFS, according to RECIST 1.1 per BICR assessment and/or locally by pathology testing, is defined as the time from the date of randomization until the date of one of the following events, whichever occurs first: (1) first documented progression that precludes surgery or requires non-protocol therapy; (2) first evidence of documented recurrence of disease post-surgery in patients who had margin-negative surgery (R0); (3) first evidence of documented progression of disease post-surgery in patients with margin-positive surgery (R1/R2); or (4) death due to any cause.

For non-RECIST 1.1 EFS determination, the EFS event date is as follows: (1) for progression that requires non-protocol therapy: the date of determination that non protocol therapy is required; (2) for progression discovered upon attempting surgery: the date of the first attempt at surgery; (3) for progression discovered during the adjuvant period: the initial date of detection (not the dates of confirmation biopsies or scans); or (4) for death: the date of death.

A recurrence of disease includes local recurrence of GC/GEJC or distant metastasis of GC/GEJC. The date of recurrence post resection is defined as the date of the initial detection of a new lesion and not the dates of confirmation biopsies or scans.

Patients are followed until an event regardless of whether the patient withdraws from therapy or receives another anticancer therapy prior to an event. Patients who do not experience an event by the time of analysis are censored at the date of their last evaluable RECIST 1.1 assessment. However, if the patient progresses or experiences recurrent disease or dies after 2 or more missed visits, the patient is censored at the time of the latest evaluable RECIST 1.1 assessment prior to the 2 or more missed visits. If the patient has no evaluable visits or does not have baseline data prior to neoadjuvant treatment, they are censored at randomization date unless they die within 2 visits of baseline for which they are treated as having an event at the death date. If the patient has surgery and there is no scan within the window following the date of surgery and prior to the start of adjuvant therapy, they are censored at the date of surgery.

The EFS time is always derived based on assessment dates and not on visit dates. EFS time equals date of EFS event or censoring-date of randomization+1.

### Overall Survival

OS is defined as the time from the date of randomization until date of death due to any cause, regardless of whether the patient withdraws from randomized therapy or receives another anti cancer therapy. Any patient not known to have died at the time of analysis is censored based on the last recorded date on which the patient was known to be alive. OS time equals date of OS event or censoring—date of randomization+1.

### Pathological Complete Response Rate

pCR is determined by pathology review. pCR rate is defined as the proportion of patients who have no residual viable tumor in the resected specimens and as determined by pathology review.

### Surgery Rate

Surgery status is determined by a patient receiving gastrectomy or gastroesophagectomy (yes/no). Surgery rate is defined as the proportion of patients who undergo gastrectomy or gastroesophagectomy.

### R0 Resection Rate

R0 resection is determined by pathology review. R0 resection rate is defined as the proportion of patients who have a confirmed complete resection (i.e., received margin-negative surgery [R]) as assessed by pathology review.

### Metastasis-Free Survival

MFS, according to RECIST 1.1, is defined as the time from the date of randomization until the date of first evidence of metastasis or death due to any cause, whichever occurs first. Patients who are alive and free from metastases at the time of analysis are censored based on the last recorded date on which the patient was known to be alive without the MFS event. MFS time equals date of MFS event or censoring-date of randomization+1.

### Disease-Specific Survival

DSS is defined as the time from the date of randomization until date of death due to GC/GEJC. Any patient not known to have died from GC/GEJC at the time of analysis is censored based on the last recorded date on which the patient was known to be alive. DSS time equals date of DSS event or censoring-date of randomization+1.

### Disease-Free Survival

DFS, according to RECIST 1.1, is defined as the time from the date of first post-surgery scan (i.e., Adjuvant Baseline scan) until the date of first evidence of recurrence of disease or death due to any cause, whichever occurs first, post-surgery in patients who had margin-negative surgery (R0). Patients who are alive and free from recurrence of disease at the time of analysis are censored based on the last recorded date on which the patient was known to be alive without the DFS event. DFS time equals date of DFS event or censoring-date of randomization+1.

### Proportion of Patients Alive/Alive and Event Free/Alive and Disease Free

The proportion of patients alive and event free at 24 months (EFS24) is defined as the Kaplan-Meier estimate of EFS (according to RECIST 1.1 per BICR assessment and/or locally by pathology testing) at 24 months after randomization.

The proportion of patients alive and event free at 36 months (EFS36) is defined as the Kaplan-Meier estimate of EFS (according to RECIST 1.1 per BICR assessment and/or locally by pathology testing) at 36 months after randomization.

The proportion of patients alive at 24 months (OS24) is defined as the Kaplan-Meier estimate of OS at 24 months after randomization.

The proportion of patients alive at 36 months (OS36) is defined as the Kaplan-Meier estimate of OS at 36 months after randomization.

The proportion of patients alive and disease free at 24 months (DFS24) is defined as the Kaplan-Meier estimate of DFS (according to RECIST 1.1) at 24 months after post-surgery scan (i.e., Adjuvant Baseline scan).

The proportion of patients alive and disease free at 36 months (DFS36) is defined as the Kaplan-Meier estimate of DFS (according to RECIST 1.1) at 36 months after post-surgery scan (i.e., Adjuvant Baseline scan).

### Adverse Events

Safety and tolerability are assessed in terms of AEs (including SAEs), deaths, laboratory data, vital signs, ECGs, and exposure. These are collected for all patients. Data from all cycles of treatment are combined in the presentation of safety data. “On treatment” is defined as assessments between date of start dose and 90 days following discontinuation of study treatment (e.g., the last dose of durvalumab). For AEs, on treatment (or TEAEs) will be defined as any AEs that started after dosing or prior to dosing and which worsen following exposure to the treatment.

### EORTC QLQ-C30 and QLQ-STO22+IL38

The EORTC QLQ-C30 is scored according to the EORTC QLQ-C30 Scoring Manual (Fayers et al., 2001). An outcome variable consisting of a score from 0 to 100 is derived for each of the symptom scales, each of the functional scales, and the global measure of health status scale in the EORTC QLQ-C30 according to the EORTC QLQ-C30 Scoring Manual. Higher scores on the global measure of health status and functional scales indicate better health status/function, but higher scores on symptom scales represent greater symptom severity. For each subscale, if <50% of the subscale items are missing, then the subscale score is divided by the number of non-missing items and multiplied by the total number of items on the subscales (Osoba et al., 1998). If at least 50% of the items are missing, then that subscale is treated as missing. Missing single items are treated as missing. The reason for any missing questionnaire is identified and recorded.

The QLQ-STO22 is scored according to its published scoring information (Blazeby et al., 2004). General principles for QLQ-STO22 scoring are identical to that for the symptom scales/single items of the EORTC QLQ-C30. Same principles are leveraged to score IL38. Higher scores in QLQ-STO22 and IL38 represent greater symptom severity/problems.

Definition of clinically meaningful changes-EORTC QLQ-C30 and QLQ-STO22+IL38: Changes in score compared with baseline are evaluated. A clinically meaningful change is defined as an absolute change in the score from baseline of >10 for scales from the EORTC QLQ-C30 and QLQ-STO22+IL38 (Osoba et al., 1998). For example, a clinically meaningful deterioration in physical function (as assessed by EORTC QLQ-C30) is defined as decrease in the score from baseline of ≥10. Similarly, a clinically meaningful deterioration or worsening in pain (as assessed by QLQ STO22) is defined as an increase in the score from baseline of ≥10. At each post baseline assessment, the change in global health status/QoL, symptoms, and functioning score from baseline will be categorized as improvement, no change, or deterioration as shown in Table 5.

TABLE 5
Mean Change and Clinically Meaningful Change-
EORTC QLQ-C30 and QLQ-STO22 + IL38

| Score | Change from baseline | Visit response |
|---|---|---|
| EORTC QLQ-C30 | ≥+10 | Improvement |
| global quality-of-life score | (increase of at least 10) | |
| | ≥−10 | Deterioration |
| | (decrease of at least 10) | |
| | Otherwise | Stable |
| EORTC QLQ-C30 | ≥+10 | Improvement |
| functional score | (increase of at least 10) | |
| | ≥−10 | Deterioration |
| | (decrease of at least 10) | |
| | Otherwise | Stable |
| EORTC QLQ-C30 and | ≥+10 | Deterioration |
| QLQ-STO22 + IL38 | (increase of at least 10) | |
| symptom scales and items | ≥−10 | Improvement |
| | (decrease of at least 10) | |
| | Otherwise | Stable |

EORTC European Organization for Research and Treatment of Cancer; IL38 Item Library 38, 2-item disease specific measure to complement; QLQ-C30 30-item core quality-of-life questionnaire; QLQ-STO22; QLQ-STO22 22-Item Gastric Cancer-Specific Quality-of-Life Questionnaire.

Time to global health status/QOL, function, and symptom deterioration, based on the clinically meaningful cutoffs, is evaluated. Change from baseline is also evaluated. Additional analyses and data visualizations may be considered.

### PRO-CTCAE

The PRO-CTCAE consists of nominal categories (e.g., “none” to “very severe” for some items in the questionnaire). Similarly, PGI-TT consists of nominal categories (e.g., “not at all” to “very much”).

### Patient Global Assessments (PGIS, PGIC, PGI-TT)

Patient global assessment data is presented using summaries and descriptive statistics. Additionally, patient global assessment data may be further explored.

### EQ-5D-5L

The EQ-5D-5L questionnaire comprises 5 questions that cover 5 dimensions of health (e.g., mobility, self-care, usual activities, pain/discomfort, and anxiety/depression) and a EQ VAS (ranging from 0 [worst imaginable health] to 100 [best imaginable health]). For each dimension, respondents select which statement best describes their health on that day from 5 possible options of increasing levels of severity (e.g., no problems, slight problems, moderate problems, severe problems, and unable to/extreme problems).

A unique EQ-5D health state, termed the EQ-5D-5L profile, is reported as a 5-digit code with a possible 3125 health states based on these responses. For example, state 11111 indicates no problems on any of the 5 dimensions. This EQ-5D-5L profile is converted into a weighted health state utility value, termed the EQ-5D index, by applying a country-specific equation to the profile that represents the comparative value of health states. This equation is based on national valuation sets elicited from the general population, and the base case is the UK perspective. Where a valuation set has not been published, the EQ-5D-5L profile is converted to the EQ 5D index using a crosswalk algorithm (Van Hout et al., 2012).

The evaluable population comprises a subset of patients in the FAS who have a baseline EQ-5D-5L assessment.

### Imaging Modalities and Acquisition Specifications for RECIST 1.1:

A summary of the imaging modalities that can be used for tumor assessment of Target Lesions (TLs), Non-Target Lesions (NTLs), and New Lesions (NLs) is provided in Table 6.

TABLE 6
Summary of Imaging Modalities for Tumor Assessment

| Target | Non-Target | |
|---|---|---|
| Lesions | Lesions | New Lesions |
| CT | CT | CT |
| MRI | MRI | MRI |
| | Plain X-ray | Plain X-ray |
| | Chest X-ray | Chest X-ray |
| | | Bone scan (Scintigraphy) |
| | | FDG-PET/CT |

CT Computed tomography; FDG-PET/CT 18F-Fluoro-deoxyglucose positron emission tomography/CT; MRI Magnetic resonance imaging.

### CT and MRI

Computed tomography (CT) with intravenous (IV) contrast is the preferred imaging modality (although magnetic resonance imaging [MRI] with IV contrast is acceptable if CT is contraindicated) to generate reproducible anatomical images for tumor assessments (i.e., for measurement of TLs, assessment of NTLs, and identification of NLs). It is essential that the same correct imaging modality, image acquisition parameters (e.g., anatomic coverage, imaging sequences), imaging facility, tumor assessor (e.g., radiologist), and method of tumor assessment (e.g., RECIST 1.1) are used consistently for each patient throughout the study. The use of the same scanner for serial scans is recommended, if possible.

Due to its inherent rapid acquisition (seconds), CT is the imaging modality of choice. Body scans should be performed with breath-hold scanning techniques, if possible. Therefore, CT of the chest is recommended over MRI due to significant motion artifacts (e.g., heart, major blood vessels, breathing) associated with MRI. MRI has excellent contrast and spatial and temporal resolutions; however, there are many image acquisition variables involved in MRI, which greatly impact image quality, lesion conspicuity, and measurement. Furthermore, the availability of MRI is variable globally. The modality used at follow-up should be the same as was used at baseline, and the lesions should be measured/assessed on the same pulse sequence. In general, local oncology diagnostic imaging parameters are applied for scan acquisition. It is beyond the scope of this appendix to prescribe specific MRI pulse sequence parameters for all scanners, body parts, and diseases.

The most critical CT and MRI image acquisition parameters for optimal tumor evaluation are anatomic coverage, contrast administration, slice thickness, and reconstruction interval.

TABLE 7
RECIST 1.1 Evaluation of Target Lesions

| | |
|---|---|
| Complete | Disappearance of all TLs since baseline. |
| response (CR) | Any pathological lymph nodes selected as TLs |
| | must have a reduction in short axis |
| | diameter to <10 mm. |
| Partial | At least a 30% decrease in the sum of the diameters of |
| response (PR) | TL, taking as reference the baseline sum of diameters. |
| Stable | Neither sufficient decrease in the sum of diameters to |
| disease (SD) | qualify for PR nor sufficient increase to qualify for PD. |
| Progression | At least a 20% increase in the sum of diameters |
| of disease (PD) | of TLs, taking as reference the smallest previous sum |
| | of diameters (nadir)-This includes the baseline |
| | sum if that is the smallest on study. |
| | In addition to the relative increase of 20%, the sum |
| | must demonstrate an absolute increase of at |
| | least 5 mm from nadir. |
| Not | Only relevant if any of the TLs at follow-up |
| evaluable (NE) | were not assessed or not evaluable (e.g., missing |
| | anatomy) or had a lesion intervention at this visit. |
| | Note: If the sum of diameters meets |
| | the progressive disease criteria, progressive disease |
| | overrides not evaluable as a TL response. |
| Not | Only relevant if no TLs present at baseline. |
| applicable (NA) | |

### RECIST 1.1 NTL Assessment at Follow-Up

All other lesions (or sites of disease) not recorded as TLs should be identified as NTLs at baseline. Measurements are not required for these lesions, but their status should be followed at subsequent visits. At each visit, an overall assessment of the NTL response should be recorded by the Investigator.

To achieve ‘unequivocal progression’ on the basis of NTLs, there must be an overall level of substantial worsening in non-target disease such that, even in presence of stable disease or partial response in TLs, the overall tumor burden has increased sufficiently to merit unequivocal progression by NTLs. A modest ‘increase’ in the size of 1 or more NTLs is usually not sufficient to qualify for unequivocal progression status. The designation of overall progression solely on the basis of change in non-target disease in the face of stable disease (SD) or progressive disease (PR) of target disease will therefore be extremely rare.

TABLE 8
RECIST 1.1 Evaluation of Non-Target Lesions

| | |
|---|---|
| Complete | Disappearance of all NTLs since baseline. |
| response (CR) | All lymph nodes must be non-pathological |
| | in size (<10 mm short axis). |
| Non CR/non PD | Persistence of 1 or more NTLs. |
| Progression (PD) | Unequivocal progression of existing NTLs. |
| | Unequivocal progression may be due to an important |
| | progression in 1 lesion only or in several |
| | lesions. In all cases, the progression |
| | MUST be clinically significant for the physician to |
| | consider changing (or stopping) therapy. |
| Not | Only relevant when 1 or some of |
| evaluable (NE) | the NTLs were not assessed and, in the Investigator's |
| | opinion, they are not able to provide an evaluable |
| | overall NTL assessment at this visit. |
| | Note: For patients without TLs at baseline, |
| | this is relevant if any of the NTLs were not |
| | assessed at this visit and the progression criteria |
| | have not been met. |
| Not | Only relevant if no NTLs present at baseline |
| applicable (NA) | |

CR Complete response; NE Not evaluable; NTL Non-target lesion; PD Progression of disease; TL Targetlesion.

### Results

Interim analysis was conducted after all randomised patients underwent or were precluded from surgery. Superiority of pCR rate (α=0.1% [2-sided]) by central review was assessed. Surgical and safety outcomes were also assessed.

For the interim analysis, there were 474 patients randomized to each treatment arm. Baseline characteristics were balanced between arms; 19% of patients in each enrolled in Asia. The majority of patients had GC (68%), cT3 (66%; cT4, 25%) and cLN+ (70%).

A statistically significant improvement in pCR was observed with addition of D to FLOT versus Placebo (19% versus 7%; A12%; odds ratio [OR], 3.08; p<0.00001; Table 9). Combined pCR/near-complete pathological response (pnCR) rate was 27% with durvalumab versus 14% with Placebo. Surgery rate and R0 resection rate (in patients who underwent surgery) were similar with durvalumab (87% and 86%, respectively) versus placebo (84% and 86%, respectively). Downstaging favored durvalumab versus placebo (pT0, 21% versus 10%; pN0, 47% versus 33%; by central review). Median durvalumab and placebo exposure was similar. Adverse event rates were similar between arms. The data demonstrate a 12% increase in pCR rate from addition of durvalumab to FLOT, and that >1 in 4 patients achieved a major response on Durva+FLOT chemotherapy, a 12.4% increase versus Placebo+FLOT chemotherapy alone (see Table 9, Table 10, and Table 11).

TABLE 9
Results

| | D + FLOT | | P + FLOT | |
|---|---|---|---|---|
| | (n = 474) | | (n = 474) | |
| pCR | | | | |
| n; % (95% CI) | 91; 19.2 | (15.8-23.0) | 34; 7.2 | (5.0-9.9) |
| OR (95% CI) | 3.08 (2.03-4.67); p < 0.00001 | | | |
| pCR/pnCR | | | | |
| n; % (95% CI) | 127; 26.8 | (22.9-31.0) | 68; 14.3 | (11.3-17.8) |
| OR (95% CI) | 2.19 (1.58-3.04); p < 0.00001 | | | |
| Surgery | | | | |
| performed | | | | |
| n; % (95% CI) | 411; 86.7 | (83.3-89.6) | 399; 84.2 | (80.6-87.4) |
| OR (95% CI) | 1.23 (0.85-1.76) | | | |
| R0 resection* | | | | |
| n; % (95% CI) | 369; 85.8 | (82.2-89.0) | 362; 85.8 | (82.1-89.0) |
| OR (95% CI) | 1.00 (0.68-1.48) | | | |
| AE^{†}, n (%) | 470 | (98.9) | 463 | (98.7) |
| Grade 3/4 | 326 | (68.6) | 317 | (67.6) |
| TRAE | 452 | (95.2) | 441 | (94.0) |
| Grade 3/4 | 275 | (57.9) | 264 | (56.3) |

TRAE

*In pts with surgery: D + FLOT, n = 430, P + FLOT, n = 422.
^{†}In safety analysis set: D + FLOT, n = 475, P + FLOT, n = 469.
TRAE, treatment-related AE.
D = Durvalumab
P = Placebo
FLOT = 5-FU + leucovorin + oxaliplatin + docetaxel

TABLE 10
Pathological Complete Response (pCR) as Assessed by Central or Local Review

| | | | | | | |
|---|---|---|---|---|---|---|
| | | | Response | Difference in | Odds | |
| | | N | rate % | response | ratio | 2-sided |
| Treatment | n | Responses ^{a} | (95% CI) ^{b} | rate (%) ^{c} | (95% CI) ^{d} | p-valued ^{d} |

pCR - Central Review (BICR)

| | | | | | | |
|---|---|---|---|---|---|---|
| Durva + | 474 | 91 | 19.2 | 12.0 | 3.08 | <0.00001 |
| FLOT | | | (15.75, 23.04) | (7.82, 16.36) | (2.03, 4.67) | |
| Placebo + | 474 | 34 | 7.2 | | | |
| FLOT | | | (5.02, 9.88) | | | |

pCR - Investigator Assessment

| | | | | | | |
|---|---|---|---|---|---|---|
| Durva + | 474 | 103 | 21.7 | 13.3 | 3.03 | <0.00001 |
| FLOT | | | (18.10, 25.72) | (8.84, 17.84) | (2.05, 4.48) | |
| Placebo + | 474 | 40 | 8.4 | | | |
| FLOT | | | (6.10, 11.31) | | | |

^{a} Patients achieve pCR if there is no residual viable tumor cells found at primary tumor and resected lymph nodes at the time of resection, meaning a pathological regression of − 100%, based on central (or local) assessment.
^{b} CIs for response rate are calculated using Clopper Pearson exact method.
^{c} Difference in response rate Durva + FLOT − Placebo + FLOT. CIs calculated using Miettinen and Nurminen's method. A difference > 0 favors durvalumab.
^{d} The analysis was performed using a stratified Cochran-Mantel-Haenszel test. The stratification factors include geographic region, clinical lymph node status and PD-L1 expression status at randomization. An Odds ratio > 0 favors durvalumab.
Durva = durvalumab
FLOT = 5-FU + leucovorin + oxaliplatin + docetaxel

TABLE 11
Major Pathological Response Rate (Complete or Near-Complete Response^{d})

| | | | Response | Difference in | Odds | |
|---|---|---|---|---|---|---|
| | | N | rate % | response | ratio | 2-sided |
| Treatment | n | Responses ^{a} | (95% CI) ^{b} | rate (%) ^{c} | (95% CI) ^{d} | p-valued ^{d} |
| Durva + | 474 | 127 | 26.8 | 12.4 | 2.19 | <0.00001 |
| FLOT | | | (22.86, 31.02) | (7.35, 17.55) | (1.58, 3.04) | |
| Placebo + | 474 | 68 | 14.3 | | | |
| FLOT | | | (11.32, 17.83) | | | |

^{a} A subject has a response if they have complete or near complete response as determined by pathology review, based on central assessment.
^{b} CIs for response rate are calculated using Clopper Pearson exact method.
^{c} Difference in response rate Durva + FLOT − Placebo + FLOT. CIs calculated using Miettinen and Nurminen's method. A difference > 0 favors durvalumab.
^{d} The analysis was performed using a stratified Cochran-Mantel-Haenszel test. The stratification factors include geographic region, clinical lymph node status and PD-L1 expression status at randomization. An Odds ratio > 0 favors durvalumab.
Near-complete response = Single cells or rare small groups of cancer cells, per modified Ryan scoring system.
Durva = durvalumab
FLOT = 5-FU + leucovorin + oxaliplatin + docetaxel

This demonstrates that perioperative durvalumab plus neoadjuvant-adjuvant chemotherapy (particularly neoadjuvant treatment) results in significant improvement of both pathological complete response (pCR) and combined near-complete pathological complete response (pnCR) among patients with resectable GC and/or resectable GEJC.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

### Embodiments

Embodiment 1: A method of treating a patient identified as having resectable gastric cancer (GC) and/or gastroesophageal junction cancer (GEJC), the method comprising administering to the patient a combination therapy comprising durvalumab and a chemotherapy. Embodiment 2: The method of embodiment 1, wherein the combination therapy comprises administering durvalumab on Day 1 and administering the chemotherapy on Day 1 and Day 15, wherein the combination therapy is administered about every 3 to 5 weeks.

Embodiment 3: The method of either embodiment 1 or embodiment 2, wherein the combination therapy comprises administering durvalumab on Day 1 and administering the chemotherapy on Day 1 and Day 15, wherein the combination therapy is administered every 4 weeks.

Embodiment 4: The method of either embodiment 2 or embodiment 3, wherein the combination therapy is administered for about 2 cycles.

Embodiment 5: The method of any one of embodiments 1 to 4 further comprising resecting the GC and/or the GEJC after administration of the combination therapy.

Embodiment 6: The method of either embodiment 4 or embodiment 5 further comprising resecting the GC and/or the GEJC about 4 weeks to about 8 weeks after the about 2 cycles of the combination therapy.

Embodiment 7: The method of either embodiment 5 or embodiment 6 further comprising administering to the patient an adjuvant combination therapy about 4 weeks to about 12 weeks after resecting the GC and/or the GEJC.

Embodiment 8: The method of embodiment 7, wherein the adjuvant combination therapy comprises administering durvalumab on Day 1 and administering the chemotherapy on Day 1 and Day 15, wherein the adjuvant combination therapy is administered about every 3 to 5 weeks.

Embodiment 9: The method of embodiment 8, the adjuvant combination therapy is administered for about 2 cycles.

Embodiment 10: The method of any one of embodiments 7 to 9, wherein the adjuvant combination therapy further comprises administering to the patient durvalumab about every 28 days (Q4W).

Embodiment 11: The method of any one of embodiments 7 to 10, wherein the adjuvant combination therapy further comprises administering to the patient durvalumab about every 28 days (Q4W), for up to about 10 cycles.

Embodiment 12: The method of any one of embodiments 1 to 11, wherein the combination therapy and/or the adjuvant combination therapy comprises about 1000 to 2000 mg of durvalumab.

Embodiment 13: The method of any one of embodiments 1 to 12, wherein the combination therapy and/or the adjuvant combination therapy comprises about 1500 mg of durvalumab.

Embodiment 14: A method of treating a patient identified as having resectable gastric cancer (GC) and/or gastroesophageal junction cancer (GEJC), the method comprising:
(i) administering to the patient, about every 4 weeks (Q4W), about 1500 mg of durvalumab on Day 1 and a chemotherapy on Day 1 and Day 15;
(ii) removing the GC and/or the GEJC by surgery;
(iii) administering to the patient, about every 4 weeks (Q4W), about 1500 mg of durvalumab on Day 1 and a chemotherapy on Day 1 and Day 15; and
(iv) administering to the patient about 1500 mg of durvalumab about every 4 weeks (Q4W).

Embodiment 15: The method of embodiment 14, wherein step (i) is administered for about 2 cycles, wherein step (iii) is administered for about 2 cycles, and/or wherein step (iv) is administered for at least about 10 cycles.

Embodiment 16: The method of any one of embodiments 1 to 15, wherein the chemotherapy is one or more of fluorouracil, leucovorin, oxaliplatin, and docetaxel.

Embodiment 17: The method of embodiment 16, wherein the chemotherapy comprises:
a fluorouracil dose of about 2000 to about 3000 mg/m²,
a leucovorin dose of about 175 to about 225 mg/m²,
an oxaliplatin dose of about 50 to about 100 mg/m², or
a docetaxel dose of about 25 to about 75 mg/m².

Embodiment 18: The method of either embodiment 16 or embodiment 17, wherein the chemotherapy comprises:
a fluorouracil dose of about 2600 mg/m²,
a leucovorin dose of about 200 mg/m²,
an oxaliplatin dose of about 85 mg/m², or
a docetaxel dose of about 50 mg/m².

Embodiment 19: The method of any one of embodiments 16 to 18, wherein the chemotherapy comprises fluorouracil, leucovorin, oxaliplatin, and docetaxel.

Embodiment 20: The method of any one of embodiments 16 to 19, wherein the chemotherapy comprises:
a fluorouracil dose of about 2600 mg/m²,
a leucovorin dose of about 200 mg/m²,
an oxaliplatin dose of about 85 mg/m², and
a docetaxel dose of about 50 mg/m².

Embodiment 21: The method of any one of embodiments 1 to 20, wherein the patient PD-L1 expression status has a TIP/TAP score ≥1%, is lymph node positive for cancer metastasis, and/or has high microsatellite instability (MSI-high).

Embodiment 22: The method of any one of embodiments 4 to 21, wherein resecting the GC and/or the GEJC occurs within about 4 weeks to about 8 weeks after the about 2 cycles of the combination therapy.

Embodiment 23: The method of any one of embodiments 1 to 22, wherein the method results in one or more of:
(i) improvement in pathological complete response (pCR) by at least about 12%;
(ii) improvement in combined pathological complete response/near-complete pathological response (pnCR) by at least about 13%;
(iii) improvement in event-free survival (EFS); and
(iv) improvement in overall survival (OS).

Embodiment 24: The method of any one of embodiments 1 to 23, wherein the method results in improvement in one or more of pCR, pnCR, EFS, and OS compared to a standard of care.

Embodiment 25: The method of any one of embodiments 1 to 23, wherein the method results in improvement in one or more of pCR, pnCR, EFS, and OS compared to a neoadjuvant-adjuvant and/or adjuvant alone FLOT.

Embodiment 26: A combination therapy comprising durvalumab and a chemotherapy for use in treating resectable gastric cancer (GC) and/or gastroesophageal junction cancer (GEJC) in a patient in need thereof.

Embodiment 27: The combination therapy for use of embodiment 26, wherein the combination therapy comprises administering durvalumab on Day 1 about every 3 to 5 weeks and administering the chemotherapy on Day 1 and Day 15 about every 3 to 5 weeks.

Embodiment 28: The combination therapy for use of embodiment 27, wherein the combination therapy is administered about every 3 to 5 weeks for about 2 cycles.

Embodiment 29: The combination therapy for use of embodiment 26, wherein the combination therapy comprises administering durvalumab on Day 1 about every 28 days (Q4W).

Embodiment 30: The combination therapy for use of either embodiment 26 or embodiment 29, wherein the combination therapy comprises administering the chemotherapy on Day 1 and Day 15 about every 28 days (Q4W).

Embodiment 31: The combination therapy for use of either embodiment 29 or embodiment 30, wherein the combination therapy is administered about every 28 days (Q4W) for about 2 cycles.

Embodiment 32: The combination therapy for use of any one of embodiments 26 to 31 further comprising resecting the GC and/or the GEJC after administration of the combination therapy.

Embodiment 33: The combination therapy for use of either embodiment 28 or embodiment 31 further comprising resecting the GC and/or the GEJC about 4 weeks to about 8 weeks after the about 2 cycles of the combination therapy.

Embodiment 34: The combination therapy for use of either embodiment 32 or embodiment 33 further comprising administering to the patient an adjuvant combination therapy about 4 weeks to about 12 weeks after resecting the GC and/or the GEJC.

Embodiment 35: The combination therapy of embodiment 34, wherein the adjuvant combination therapy comprises administering durvalumab on Day 1 about every 3 to 5 weeks.

Embodiment 36: The combination therapy for use of either embodiment 34 or embodiment 35, wherein the adjuvant combination therapy comprises administering the chemotherapy on Day 1 and Day 15 about every 3 to 5 weeks.

Embodiment 37: The combination therapy for use of either embodiment 35 or embodiment 36, wherein the adjuvant combination therapy is administered about every 3 to 5 weeks for about 2 cycles.

Embodiment 38: The combination therapy for use of any one of embodiments 34 to 37, wherein the adjuvant chemotherapy further comprises administering to the patient durvalumab about every 28 days (Q4W) for up to about 10 cycles.

Embodiment 39: The combination therapy for use of any one of embodiments 26 to 38, wherein the combination therapy and/or the adjuvant combination therapy comprises about 1000 to 2000 mg of durvalumab.

Embodiment 40: The combination therapy for use of any one of embodiments 26 to 38, wherein the combination therapy and/or the adjuvant combination therapy comprises about 1500 mg of durvalumab.

Embodiment 41: A combination therapy for use in treating a patient identified as having resectable gastric cancer (GC) and/or gastroesophageal junction cancer (GEJC), wherein the combination therapy comprises:
(i) administering to the patient, about every 4 weeks (Q4W), about 1500 mg of durvalumab on Day 1 and a chemotherapy on Day 1 and Day 15;
(ii) removing the GC and/or the GEJC by surgery;
(iii) administering to the patient, about every 4 weeks (Q4W), about 1500 mg of durvalumab on Day 1 and a chemotherapy on Day 1 and Day 15; and
(iv) administering to the patient about 1500 mg of durvalumab about every 4 weeks (Q4W).

Embodiment 42: The combination therapy for use of embodiment 41, wherein step (i) is administered for about 2 cycles, wherein step (iii) is administered for about 2 cycles, and/or wherein step (iv) is administered for at least about 10 cycles.

Embodiment 43: The combination therapy for use of any one of embodiments 26 to 42, wherein the chemotherapy is one or more of fluorouracil, leucovorin, oxaliplatin, and docetaxel.

Embodiment 44: The combination therapy for use of embodiment 43, wherein the chemotherapy comprises:
a fluorouracil dose of about 2000 to about 3000 mg/m²,
a leucovorin dose of about 175 to about 225 mg/m²,
an oxaliplatin dose of about 50 to about 100 mg/m², or
a docetaxel dose of about 25 to about 75 mg/m².

Embodiment 45: The combination therapy for use of either embodiment 43 or embodiment 44, wherein the chemotherapy comprises:
a fluorouracil dose of about 2600 mg/m²,
a leucovorin dose of about 200 mg/m²,
an oxaliplatin dose of about 85 mg/m², or
a docetaxel dose of about 50 mg/m².

Embodiment 46: The combination therapy for use of any one of embodiments 43 to 45, wherein the chemotherapy comprises fluorouracil, leucovorin, oxaliplatin, and docetaxel.

Embodiment 47: The combination therapy for use of any one of embodiments 43 to 46, wherein the chemotherapy comprises:
a fluorouracil dose of about 2600 mg/m²,
a leucovorin dose of about 200 mg/m²,
an oxaliplatin dose of about 85 mg/m², and
a docetaxel dose of about 50 mg/m².

Embodiment 48: The combination therapy for use of any one of embodiments 26 to 47, wherein the patient PD-L1 expression status has a TIP/TAP score ≥1%, is lymph node positive for cancer metastasis, and/or has high microsatellite instability (MSI-high).

Embodiment 49: The combination therapy for use of any one of embodiments 32 to 48, wherein resecting the GC and/or the GEJC occurs within about 4 weeks to about 8 weeks after the about 2 cycles of the combination therapy.

Embodiment 50: The combination therapy for use of any one of embodiments 26 to 49, wherein the use of the combination therapy results in one or more of:
(i) improvement in pathological complete response (pCR) by at least about 12%;
(ii) improvement in combined pathological complete response/near-complete pathological response (pnCR) by at least about 13%;
(iii) improvement in event-free survival (EFS); and
(iv) improvement in overall survival (OS).

Embodiment 51: The combination therapy for use of any one of embodiments 26 to 50, wherein the use of the combination therapy results in improvement in one or more of pCR, pnCR, EFS, and OS compared to a standard of care.

Embodiment 52: The combination therapy for use of any one of embodiments 26 to 51, wherein the use of the combination therapy results in improvement in one or more of pCR, pnCR, EFS, and OS compared to a neoadjuvant-adjuvant and/or adjuvant alone FLOT. Embodiment 53: Use of a combination therapy comprising durvalumab and a chemotherapy for the manufacture of a medicament for treating resectable gastric cancer (GC) and/or gastroesophageal junction cancer (GEJC) in a patient in need thereof.

Embodiment 54: The use of embodiment 53, wherein the medicament is administered about every 3 to 5 weeks and the combination therapy comprises administering durvalumab on Day 1 and administering the chemotherapy on Day 1 and Day 15.

Embodiment 55: The use of embodiment 53, wherein the medicament is administered about every 28 days (Q4W) and the combination therapy comprises administering durvalumab on Day 1 and administering the chemotherapy on Day 1 and Day 15.

Embodiment 56: The use of either embodiment 54 or embodiment 55, wherein the combination therapy is administered for about 2 cycles.

Embodiment 57: The use of any one of embodiments 53 to 56 further comprising resecting the GC and/or the GEJC after administration of the combination therapy.

Embodiment 58: The use of either embodiment 56 or embodiment 57 further comprising resecting the GC and/or the GEJC about 4 weeks to about 8 weeks after the about 2 cycles of the combination therapy.

Embodiment 59: The use of either embodiment 57 or embodiment 58 further comprising administering to the patient an adjuvant combination therapy about 4 weeks to about 12 weeks after resecting the GC and/or the GEJC.

Embodiment 60: The use of embodiment 59, wherein the adjuvant combination therapy comprises administering durvalumab on Day 1 about every 3 to 5 weeks.

Embodiment 61: The use of embodiment 59, wherein the adjuvant combination therapy comprises administering the chemotherapy on Day 1 and Day 15 about every 3 to 5 weeks.

Embodiment 62: The use of either embodiment 60 or embodiment 61, wherein the adjuvant combination therapy is administered about every 3 to 5 weeks for about 2 cycles.

Embodiment 63: The use of any one of embodiments s 59 to 62, wherein the adjuvant combination therapy further comprises administering to the patient durvalumab about every 28 days (Q4W) for up to 10 cycles.

Embodiment 64: The use of any one of embodiments 53 to 63, wherein the medicament comprises about 1000 to 2000 mg of durvalumab.

Embodiment 65: The use of any one of embodiments 53 to 63, wherein the medicament comprises about 1500 mg of durvalumab.

Embodiment 66: The use of durvalumab and a chemotherapy in the manufacture of a medicament for treating resectable gastric cancer (GC) and/or gastroesophageal junction cancer (GEJC) in a combination therapy, wherein the combination therapy comprises:
(i) administering to the patient, about every 4 weeks (Q4W), about 1500 mg of durvalumab on Day 1 and a chemotherapy on Day 1 and Day 15;
(ii) removing the GC and/or the GEJC by surgery;
(iii) administering to the patient, about every 4 weeks (Q4W), about 1500 mg of durvalumab on Day 1 and a chemotherapy on Day 1 and Day 15; and
(iv) administering to the patient about 1500 mg of durvalumab about every 4 weeks (Q4W).

Embodiment 67: The use of embodiment 66, wherein step (i) is administered for about 2 cycles, wherein step (iii) is administered for about 2 cycles, and/or wherein step (iv) is administered for at least about 10 cycles.

Embodiment 68: The use of any one of embodiments 53 to 67, wherein the chemotherapy is one or more of fluorouracil, leucovorin, oxaliplatin, and docetaxel.

Embodiment 69: The use of embodiment 68, wherein the chemotherapy comprises:
a fluorouracil dose of about 2000 to about 3000 mg/m²,
a leucovorin dose of about 175 to about 225 mg/m²,
an oxaliplatin dose of about 50 to about 100 mg/m², or
a docetaxel dose of about 25 to about 75 mg/m².

Embodiment 70: The use of either embodiment 68 or embodiment 69, wherein the chemotherapy comprises:
a fluorouracil dose of about 2600 mg/m²,
a leucovorin dose of about 200 mg/m²,
an oxaliplatin dose of about 85 mg/m², or
a docetaxel dose of about 50 mg/m².

Embodiment 71: The use of any one of embodiments 68 to 70, wherein the chemotherapy comprises fluorouracil, leucovorin, oxaliplatin, and docetaxel.

Embodiment 72: The use of any one of embodiments 68 to 71, wherein the chemotherapy comprises:
a fluorouracil dose of about 2600 mg/m²,
a leucovorin dose of about 200 mg/m²,
an oxaliplatin dose of about 85 mg/m², and
a docetaxel dose of about 50 mg/m².

Embodiment 73: The use of any one of embodiments 53 to 72, wherein the patient PD-L1 expression status has a TIP/TAP≥1%, is lymph node positive for cancer metastasis, and/or has high microsatellite instability (MSI-high).

Embodiment 74: The use of any one of embodiments 53 to 73, wherein resecting the GC and/or the GEJC occurs within about 4 weeks to about 8 weeks after the about 2 cycles of combination therapy.

Embodiment 75: The use of any one of embodiments 53 to 74, wherein the use of the combination therapy results in one or more of:
(i) improvement in pathological complete response (pCR) by at least about 12%;
(ii) improvement in combined pathological complete response/near-complete pathological response (pnCR) by at least about 13%;
(iii) improvement in event-free survival (EFS); and
(iv) improvement in overall survival (OS).

Embodiment 76: The use of any one of embodiments 53 to 75, wherein the use of the combination therapy results in improvement in one or more of pCR, pnCR, EF, and/or OS compared to a standard of care.

Embodiment 77: The use of any one of embodiments 53 to 75, wherein the use of the combination therapy results in improvement in one or more of pCR, pnCR, EFS, and/or OS compared to a neoadjuvant-adjuvant and/or adjuvant alone FLOT.

### BIBLIOGRAPHY

Aaronson et al., “The European Organization for Research and Treatment of Cancer QLQ-C30: a quality-of-life instrument for use in international clinical trials in oncology,” J. Natl. Cancer Inst. 85 (5): 365-76 (1993).
Al-Batran et al., “Histopathological regression after neoadjuvant docetaxel, oxaliplatin, fluorouracil, and leucovorin versus epirubicin, cisplatin, and fluorouracil or capecitabine in patients with resectable gastric or gastro-oesophageal junction adenocarcinoma (FLOT4-AIO): results from the phase 2 part of a multicentre, open-label, randomised phase 2/3 trial,” Lancet Oncol. 17 (12): 1697-708 (2016).
Al-Batran et al., “Perioperative chemotherapy with docetaxel, oxaliplatin, and fluorouracil/leucovorin (FLOT) versus epirubicin, cisplatin, and fluorouracil or capecitabine (ECF/ECX) for resectable gastric or gastroesophageal junction (GEJ) adenocarcinoma (FLOT4-AIO): A multicenter, randomized phase 3 trial,” J. Clin. Oncol. 35 (15): 1-4 (2017).
Al-Batran et al., “Perioperative chemotherapy with fluorouracil plus leucovorin, oxaliplatin, and docetaxel versus fluorouracil or capecitabine plus cisplatin and epirubicin for locally advanced, resectable, gastric or gastro oesophageal junction adenocarcinoma (FLOT4): A randomized, phase 2/3 trial,” Lancet 393 (10184): 1948-57 and supplementary appendix (2019).
Alexandrov et al., “Signatures of mutational processes in human cancer,” Nature 500:415-21 (2013).
Amin et al., AJCC Cancer Staging Manual (8th edition), Springer International Publishing: American Joint Commission on Cancer (2017).
Antonia et al., “Durvalumab after chemoradiotherapy in Stage III Non-Small-Cell Lung Cancer,” N. Engl. J. Med. 377:1919-29 (2017).
Bang et al., “Adjuvant capecitabine and oxaliplatin for gastric cancer after D2 gastrectomy (CLASSIC): a phase 3 open-label, randomised controlled trial,” Lancet 379:315-21 (2012).
Basch et al., “Adverse symptom event reporting by patients vs clinicians: relationship with clinical outcomes,” J. Natl. Cancer Inst. 101:1624-32 (2009).
Bass et al., “Comprehensive molecular characterization of gastric adenocarcinoma,” Nature 513 (7517): 202-09 (2014).
Bayne et al., “Tumor-derived granulocyte-macrophage colony-stimulating factor regulates myeloid inflammation and T cell immunity in pancreatic cancer,” Cancer Cell 21 (6): 822-35 (2012).
Bilici, “Prognostic factors related with survival in patients with pancreatic adenocarcinoma,” World J. Gastroenterol. 20 (31): 10802-12 (2014).
Blazeby et al., “Clinical and psychometric validation of a questionnaire module, the EORTC QLQ-STO 22, to assess quality of life in patients with gastric cancer,” Eur. J. Cancer 40:2260-68 (2004).
Bouvier et al., “How many nodes must be examined to accurately stage gastric carcinomas? Results from a population based study,” Cancer 94:2862-66 (2002).
Bracci et al., “Immune-based mechanisms of cytotoxic chemotherapy: implications for the design of novel and rationale-based combined treatments against cancer,” Cell Death Differ. 21 (1): 15-25 (2014).
Brahmer et al., “Safety and activity of anti-PD-L1 antibody in patients with advanced cancer,” N. Engl. J. Med. 366 (26): 2455-65 (2012).
Cunningham et al., “Perioperative chemotherapy versus surgery alone for resectable gastroesophageal cancer,” N. Engl. J. Med. 355:11-20 (2006).
Cunningham et al., “Capecitabine and oxaliplatin for advanced esophagogastric cancer,” N. Engl. J. Med. 358:36-46 (2008).
Dunn et al., “The three Es of cancer immunoediting,” Annu. Rev. Immunol. 22:329-60 (2004).
European Medicines Agency, Guideline on the evaluation of anticancer medicinal products in man, London (2017).
Emens et al., “The interplay of immunotherapy and chemotherapy: Harnessing potential synergies,” Cancer Immunol. Res. 3 (5): 436-43 (2015).
Eun et al., “Effects of continuing adjuvant S-1 for 1 year on the prognosis of gastric cancer patients: Results from a prospective single center study,” J. Gastric Cancer 15 (2): 113-20 (2015).
EuroQol Group, EQ-5D-5L User Guide: Basic information on how to use the EQ-5D-5L instrument, Version 2.1, April 2015.
Fairman et al., “Pharmacokinetics of durvalumab, a fully human anti-PDL1 monoclonal antibody, in patients with advanced solid tumours,” J. Clin. Oncol. 2014 ASCO Annual Meeting Abstracts, 32 (5s): (suppl; abstr 2602).
Fayers et al. on behalf of the EORTC Quality of Life Study Group, EORTC QLQ-C30 Scoring Manual: 3rd Edition 2001.
FDA-NIH Biomarker Working Group. BEST (Biomarkers, EndpointS, and other Tools) Resource [Internet]. Silver Spring (MD): Food and Drug Administration (US), Glossary. (2016). Food and Drug Administration. Guidance for Industry, Clinical Trial Endpoints for the Approval of Cancer Drugs and Biologics.
Fuchs et al., KEYNOTE-059 cohort 1: Efficacy and safety of pembrolizumab (pembro) monotherapy in patients with previously treated advanced gastric cancer,” J. Clin. Oncol. 35 (15_suppl): 4003 (2017).
Gail M et al., “Tests for qualitative interactions between treatment effects and patient subsets,” Biometrics 41 (2): 361-72 (1985).
Gandhi et al., “Pembrolizumab plus Chemotherapy in Metastatic Non-Small-Cell Lung Cancer,” N. Engl. J. Med. 378:2078-92 (2018).
New Global Cancer Date: GLOBOCAN 2018.
Hato et al., “Molecular Pathways: The Immunogenic Effects of Platinum-Based Chemotherapeutics,” Clin. Cancer Res. 20 (11): 2831-37 (2014).
Hirano et al., “Blockade of B7-H1 and PD-1 by monoclonal antibodies potentiates cancer therapeutic immunity,” Cancer Res. 65 (3): 1089-96 (2005).
Huang et al., “Multivariate analysis identified nodal status (HR=4.20), vessel involvement (HR=3.40) and histological differentiation (HR=3.52) as independent prognostic factors for disease-free survival in early stage of gastric cancer,” Exp. Ther. Med. 2 (6): 1059-64 (2011).
Iwai et al., “Involvement of PD-L1 on tumor cells in the escape from host immune system and tumor immunotherapy by PD-L1 blockade,” Proc. Natl. Acad. Sci. U.S.A. 99:12293-97 (2002).
Janjigian et al., “CheckMate-032 Study: Efficacy and Safety of Nivolumab and
Nivolumab Plus Ipilimumab in Patients With Metastatic Esophagogastric Cancer,” J. Clin. Oncol. 36:2836-44 (2018).
Karpeh et al., “Lymph node staging in gastric cancer: Is location more important than number? An analysis of 1,038 patients,” Ann. Surg. 232 (3): 362-71 (2000).
Keir et al., “PD-1 and its ligands in tolerance and immunity,” Annu. Rev. Immunol. 26:677-704 (2008).
Klein et al., “Analyzing survival curves at a fixed point in time,” Stat. Med. 26 (24): 4505-19 (2007).
Kluetz et al., “Incorporating the patient experience into regulatory decision making in the USA, Europe, and Canada,” Lancet Oncol. 19 (5): e267-74 (2018).
Kluetz et al., “Informing the tolerability of cancer treatments using patient-reported outcome measures: Summary of an FDA and Critical Path Institute workshop,” Value Health 21 (6): 742-47 (2018).
Kodera et al., “A randomized phase III study comparing S-1 plus docetaxel with S-1 alone as a postoperative adjuvant chemotherapy for curatively resected stage III gastric cancer (JACCRO GC-07 trial),” J. Clin. Oncol. 36: 15_suppl, 4007 (2018).
Lan et al., “Discrete sequential boundaries for clinical trials,” Biometrika 70:659-63 (1983).
Litwin et al., “Differences in urologist and patient assessments of health related quality of life in men with prostate cancer: results of the CaPSURE database,” J. Urol. 159:1988-92 (1998).
Liu et al., “Prognostic significance of PD-L1 expression in patients with gastric cancer in East Asia: a meta-analysis,” Onco. Targets Ther. 9:2649-54 (2016).
Macdonald, “Gastric Cancer: Nagoya is not New York,” J. Clin. Oncol. 29 (33): 4348-50 (2011).
Markar et al., “Long-term survival after gastrectomy for cancer in randomized, controlled oncological trials: comparison between West and East,” Ann. Surg. Oncol. 20:2328-38 (2013).
Narwal et al., “Population pharmacokinetics of sifalimumab, an investigational anti-interferonalpha monoclonal antibody, in systemic lupus erythematosus,” Clin. Pharmacokinet. 52:1017-27 (2013).
National Comprehensive Cancer Network (NCCN), Gastric Cancer, version February 2018-May 22, 2018.
Ng et al., “Rationale for fixed dosing of pertuzumab in cancer patients based on population pharmacokinetic analysis,” Pharm. Res. 23 (6): 1275-84 (2006).
Okazaki et al., “PD-1 and PD-1 ligands: from discovery to clinical application,” Int. Immunol. 19 (7): 813-24 (2007).
Okudaira et al., “Blockade of B7 H1 or B7-DC induces an antitumor effect in a mouse pancreatic cancer model,” Int. J. Oncol. 35 (4): 741-49 (2009).
Osoba et al., “Interpreting the significance of changes in health-related quality-of-life scores,” J. Clin. Oncol. 16 (1): 139-44 (1998).
Pardoll, “The blockade of immune checkpoints in cancer immunotherapy,” Nat. Rev. Cancer 12:252-64 (2012).
Paz-Ares et al., “Phase 3 study of carboplatin-paclitaxel with or without pembrolizumab for patients with metastatic squamous non-small-cell lung cancer (NSCLC),” J. Clin. Oncol. 36 (15): 1-5 (2018).
Powles et al., “MPDL3280A (anti-PD-L1) treatment leads to clinical activity in metastatic bladder cancer,” Nature 515 (7528): 558-62 (2014).
Qin et al., “Mechanisms of immune evasion and current status of checkpoint inhibitors in non-small cell lung cancer,” Cancer Med. 9:2169-70 (2016).
Rizvi et al., “Safety and clinical activity of MEDI4736, an anti-programmed cell death-ligand-1 (PD-L1) antibody, in patients with nonsmall cell lung cancer (NSCLC),” J. Clin. Oncol. 33: Abstract 8032 (2015).
Sasako et al., “D2 lymphadenectomy alone or with para-aortic nodal dissection for gastric cancer,” N. Engl. J. Med. 359:453-62 (2008).
Sasako et al., “Five-year outcomes of a randomized Phase III trial comparing adjuvant chemotherapy with S-1 versus surgery alone in Stage II or III gastric cancer,” J. Clin. Oncol. 29 (33): 4387-93 (2011).
Segal et al., “Safety and efficacy of MEDI4736, an anti-PD-L1 antibody, in patients from a squamous cell carcinoma of the head and neck (SCCHN) expansion cohort,” J. Clin. Oncol. 33: Abstract 3011 (2015).
Siewertet et al., “Relevant prognostic factors in gastric cancer ten year results of the german gastric cancer study,” Ann. Surg. 228 (4): 449-61 (1998).
Smalley et al., “Updated Analysis of SWOG-Directed Intergroup Study 0116: A Phase III Trial of Adjuvant Radiochemotherapy Versus Observation After Curative Gastric Cancer Resection,” J. Clin. Oncol. 30 (19): 2327-33 (2012).
Smyth et al., “Gastric Cancer: ESMO Clinical Practice Guidelines,” Ann. Oncol. 27 (suppl 5): v38-v49 (2016).
Soliman, “nab-Paclitaxel as a potential partner with checkpoint inhibitors in solid tumors,” Onco. Targets Ther. 10:101-12 (2017).
Sprangers et al., “The role of health care providers and significant others in evaluating the quality of life of patients with chronic disease: a review,” J. Clin. Epidemiol. 45:743-60 (1992).
Stewart et al., “Identification and characterization of MEDI4736, an antagonistic anti-PD-L1 monoclonal antibody,” Cancer Immunol. Res. 3 (9): 1052-62 (2015).
Topalian et al., “Safety, activity, and immune correlates of anti-PD-1 antibody in cancer,” N. Engl. J. Med. 366:2443-54 (2012).
van Hout et al., “Interim scoring for the EQ-5D-5L: mapping the EQ-5D-5L to EQ-5D-3L value sets,” Value Health 15 (5): 708-15 (2012).
Vanderwalde et al., “Microsatellite instability status determined by next-generation sequencing and compared with PD-L1 and tumor mutational burden in 11,348 patients,” Cancer Med. 7 (3): 746-56 (2018).
Vanneman et al., “Combining immunotherapy and targeted therapies in cancer treatment,” Nat. Rev. Cancer 12 (4): 237-51 (2012).
Verdecchia et al., “Explaining gastric cancer survival differences among European countries,” Int. J. Cancer 109:737-41 (2004).
Vickery et al., “Development of an EORTC disease-specific quality of life module for use in patients with gastric cancer,” Eur. J. Cancer 37:966-71 (2001).
Wainberg et al., “KEYNOTE-059 update: Efficacy and safety of pembrolizumab alone or in combination with chemotherapy in patients with advanced gastroc and gastroesophageal (G/GEJ) Cancer,” Ann. Oncol. 28 (5): 616-17 (2017).
Wang et al., “Fixed dosing versus body size based dosing of monoclonal antibodies in adult clinical trials,” J. Clin. Pharmacol. 49 (9): 1012-24 (2009).
Woo et al., “Lymphadenectomy with optimum of 29 lymph nodes retrieved associated with improved survival in advanced gastric cancer: a 25,000-patient international database study,” J. Am. Coll. Surg. 224:546-55 (2017).
Wu et al., “PD-L1 and survival in solid tumors: A Meta-Analysis,” PloS One 10 (6): e0131403 (2015).
Xu et al., “Clinicopathologic Significance and Prognostic Value of B7 Homolog 1 in Gastric Cancer: A Systematic Review and Meta Analysis,” Medicine 94 (43): e1911 (2015).
Ychou et al., “Perioperative chemotherapy compared with surgery alone for resectable gastroesophageal adenocarcinoma: An FNCLCC and FFCD multicenter Phase III trial,” J. Clin. Oncol. 29 (13): 1715-21 (2011).
Zhang et al., “Antitumor immunotherapy by blockade of the PD-1/PD-L1 pathway with recombinant human PD-1-IgV,” Cytotherapy 10 (7): 711-19 (2008).
Zhang et al., “Fixed dosing versus body size-based dosing of therapeutic peptides and proteins in adults,” J. Clin. Pharmacol. 52 (1): 18-28 (2012).
Zhu et al., “Microsatellite instability and survival in gastric cancer: A systematic review and meta-analysis,” Mol. Clin. Oncol. 3 (3): 699-705 (2015).

1: A method of treating a patient identified as having resectable gastric cancer (GC) and/or gastroesophageal junction cancer (GEJC), the method comprising administering to the patient a combination therapy comprising durvalumab and a chemotherapy.

2: The method of claim 1, wherein the combination therapy comprises administering durvalumab on Day 1 and administering the chemotherapy on Day 1 and Day 15, wherein the combination therapy is administered about every 3 to 5 weeks.

3: The method of claim 1, wherein the combination therapy comprises administering durvalumab on Day 1 and administering the chemotherapy on Day 1 and Day 15, wherein the combination therapy is administered every 4 weeks.

4: The method of either claim 2, wherein the combination therapy is administered for about 2 cycles.

5: The method of claim 1 further comprising resecting the GC and/or the GEJC after administration of the combination therapy.

6: The method of claim 5 further comprising resecting the GC and/or the GEJC about 4 weeks to about 8 weeks after the about 2 cycles of the combination therapy.

7: The method of claim 6 further comprising administering to the patient an adjuvant combination therapy about 4 weeks to about 12 weeks after resecting the GC and/or the GEJC.

8: The method of claim 7, wherein the adjuvant combination therapy comprises administering durvalumab on Day 1 and administering the chemotherapy on Day 1 and Day 15, wherein the adjuvant combination therapy is administered about every 3 to 5 weeks.

9: The method of claim 8, the adjuvant combination therapy is administered for about 2 cycles.

10: The method of claim 7, wherein the adjuvant combination therapy further comprises administering to the patient durvalumab about every 28 days (Q4W).

11: The method of claim 7, wherein the adjuvant combination therapy further comprises administering to the patient durvalumab about every 28 days (Q4W), for up to about 10 cycles.

12: The method of claim 1, wherein the combination therapy and/or the adjuvant combination therapy comprises about 1000 to 2000 mg of durvalumab.

13: The method of claim 1, wherein the combination therapy and/or the adjuvant combination therapy comprises about 1500 mg of durvalumab.

14: A method of treating a patient identified as having resectable gastric cancer (GC) and/or gastroesophageal junction cancer (GEJC), the method comprising:
(i) administering to the patient, about every 4 weeks (Q4W), about 1500 mg of durvalumab on Day 1 and a chemotherapy on Day 1 and Day 15;
(ii) removing the GC and/or the GEJC by surgery;
(iii) administering to the patient, about every 4 weeks (Q4W), about 1500 mg of durvalumab on Day 1 and a chemotherapy on Day 1 and Day 15; and
(iv) administering to the patient about 1500 mg of durvalumab about every 4 weeks (Q4W).

15: The method of claim 14, wherein step (i) is administered for about 2 cycles, wherein step (iii) is administered for about 2 cycles, and/or wherein step (iv) is administered for at least about 10 cycles.

16: The method of either claim 1 or claim 14, wherein the chemotherapy is one or more of fluorouracil, leucovorin, oxaliplatin, and docetaxel.

17: The method of claim 16, wherein the chemotherapy comprises:
a fluorouracil dose of about 2000 to about 3000 mg/m²,
a leucovorin dose of about 175 to about 225 mg/m²,
an oxaliplatin dose of about 50 to about 100 mg/m², or
a docetaxel dose of about 25 to about 75 mg/m².

18: The method of claim 16, wherein the chemotherapy comprises:
a fluorouracil dose of about 2600 mg/m²,
a leucovorin dose of about 200 mg/m²,
an oxaliplatin dose of about 85 mg/m², or
a docetaxel dose of about 50 mg/m².

19: The method of claim 16, wherein the chemotherapy comprises fluorouracil, leucovorin, oxaliplatin, and docetaxel.

20: The method of claim 16, wherein the chemotherapy comprises:
a fluorouracil dose of about 2600 mg/m²,
a leucovorin dose of about 200 mg/m²,
an oxaliplatin dose of about 85 mg/m², and
a docetaxel dose of about 50 mg/m².

21: The method of either claim 1 or claim 14, wherein the patient PD-L1 expression status has a TIP/TAP score ≥1%, is lymph node positive for cancer metastasis, and/or has high microsatellite instability (MSI-high).

22: The method of either claim 5 or claim 14, wherein resecting the GC and/or the GEJC occurs within about 4 weeks to about 8 weeks after the about 2 cycles of the combination therapy.

23: The method of either claim 1 or claim 14, wherein the method results in one or more of:
(i) improvement in pathological complete response (pCR) by at least about 12%;
(ii) improvement in combined pathological complete response/near-complete pathological response (pnCR) by at least about 13%;
(iii) improvement in event-free survival (EFS); and
(iv) improvement in overall survival (OS).

24: The method of either claim 1 or claim 14, wherein the method results in improvement in one or more of pCR, pnCR, EFS, and OS compared to a standard of care; and/or compared to a neoadjuvant-adjuvant and/or adjuvant alone FLOT.

25-77. (canceled)


SEQUENCE LISTING (8 sequences)

SEQ ID NO: 1 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 12 aa, synthetic construct
RASQRVSSSYLA

SEQ ID NO: 3 - protein, 7 aa, synthetic construct
DASSRAT

SEQ ID NO: 4 - protein, 9 aa, synthetic construct
QQYGSLPWT

SEQ ID NO: 5 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 6 - protein, 10 aa, synthetic construct
GFTFSRYWMS

SEQ ID NO: 7 - protein, 17 aa, synthetic construct
NIKQDGSEKYYVDSVKG

SEQ ID NO: 8 - protein, 12 aa, synthetic construct
EGGWFGELAFDY